(19) United States
(12) Patent Application Publication
(10) Pub. No.: US 2004/0261141 A1
(43) Pub. Date: Dec. 23, 2004

(54) Animal model for therapy of diseases of the eye

(76) Inventors: Bernhard Heinrich Friedrich Weber, Gerbrunn (DE); Karina Drumm, Wurzburg (DE); Frank Gohring, Wurzburg (DE)

Correspondence Address: Cooper & Dunham LLP, 1185 Avenue of the Americas, New York, NY, 10036, US

(21) Appl. No.: 10/830,282

(22) Filed: Apr. 21, 2004

Related U.S. Application Data
(60) Provisional application No. 60/466,606, filed on Apr. 30, 2003.

(51) Int. Cl. A01K 67/027

(52) U.S. Cl. 800/18

(57) ABSTRACT

Provided are animal models for the analysis of physiological states and therapy for X-linked juvenile retinoschisis. Furthermore, methods of screening test therapies as potential prevention or treatment of retinoschisis are described as well as methods of prevention or treatment of retinoschisis making use of those therapies.

### FIELD OF THE INVENTION

The present invention relates to the technical field of genes predominantly or specifically expressed in tissue of the back of the eye, which are etiologically related to the genesis of retinal eye diseases in general and X-linked juvenile retinoschisis in particular. The present invention provides animals deficient in RS1 encoding retinoschisin gene. Also provided are methods of using the mice, and other mammals as animal models for the analysis of physiological states of and therapy for X-linked juvenile retinoschisis.

### BACKGROUND OF THE INVENTION

X-linked juvenile retinoschisis (RS) is a common cause of juvenile macular degeneration affecting approximately 300.000 young males worldwide (De la Chapelle et al. 1994). The disease is characterized by a splitting or schisis of the inner retinal layers resulting in cystic degeneration of the central retina (Condon et al. 1986, George et al. 1996). Approximately half of the patients also develop peripheral manifestations (George et al. 1996, Roesch et al. 1998). RS is clinically variable with patients typically presenting with progressive visual impairment (20/30 to 20/200), strabismus or nystagmus between 5 and 10 years of age. Severely affected persons may be blind at birth, although generally the clinical course is more benign, with only a moderate decrease in visual acuity. At later stages of the disease severe complications such as retinal detachment, vitreal hemorrhage or choroidal sclerosis may occur and may ultimately lead to blindness (Roesch et al. 1998). The brief-flash electroretinogram (ERG) of affected males exhibit normal or near normal a-wave amplitudes suggestive of preserved rod and cone photoreceptor systems but substantially reduced b-waves, indicating loss of bipolar cell activity (Robson et al. 1998). To date, there is no therapeutic treatment for RS, the retinal schisis cannot be corrected by medication or surgery.

Thus, there is a continuing need in the art for new tools to study ocular diseases and ways and means to treat and prevent this common form of macular degeneration.

### SUMMARY OF THE INVENTION

It is an object of the present invention to provide a transgenic animals, particularly mammals useful for studying retinoschisis.

It is another object of the present invention to provide a transgenic animal, in particular mouse useful for developing medicinal, radiological, surgical and gene therapies for retinoschisis.

It is an object of the present invention to provide a method for screening compounds for use in treating and preventing retinopathies.

Another object of the invention is to provide a method for screening radiological and gene therapies for use in treating and preventing retinoschisis

These and other objects of the invention are achieved by providing a transgenic non-human animal comprising a recombinant nucleic acid molecule the presence of which leads to inactivation of the expression of a gene orthologous to the human RS1 gene, and wherein said animal displays one or more clinical symptoms of X-linked juvenile retinoschisis (RS).

According to another aspect of the invention a transgenic animal, in particular mouse is provided comprising one or more nucleic acid molecules comprising a nucleotide sequence derived from the human RS1 gene or from a corresponding ortholog or a vector encoding and capable of expressing such nucleic acid molecules, wherein said nucleic acid molecule is capable of provoking the degradation of the corresponding mRNA encoding RS1 or an orthologous gene product. The animal developes one or more clinical symptoms of X-linked juvenile retinoschisis (RS). Symptoms are determined by Scanning-Laser Ophthalmoscopy, Electroretinogram, Histology and Electron Microscopy, Immunofluorescence Labeling, and/or Cone Photoreceptor Count.

In yet another embodiment of the invention a method of screening test therapies as potential therapies for preventing and treating retinoschisis is provided. A transgenic animal is subjected to a test therapy. The onset or development of one or more of the clinical symptoms of X-linked juvenile retinoschisis (RS) in the transgenic mammal is determined in order to determine the optimal time and dosage regimen for therapeutic intervention. The identified test therapy leads to potential therapy for preventing or treating retinoschisis, for example by surgical or medical treatment, and provides guidance in the formulation of drugs, in particular for defining the optimal time frame for release of the drug in the body in order to excert its effects at the appropriate time before or at the onset of the disease, thereby also circumventing potential undesired side effects. Typically, the method of the present invention for prevention or treatment of retinoschisis comprises administering to a subject in need thereof a therapeutically effective amount of a compound capable to compensate for the loss of expression of the RS1 gene or for the activity of the RS1 gene product to the subject, preferably according to a test therapy determined in accordance with the animal model of the instant invention. Alternatively, a pharmaceutical composition comprising a compound identified or isolated according to the methods described herein can be used, wherein said composition is formulated so to relase the drug at the time and/or in dosage determined with the mentioned test therapy.

The present invention thus provides the art with an extremely useful model of testing therapies for therapeutic and diagnostic approaches for retinoschisis and other occular diseases. The model is relatively cheap and reliable, does not require any exogenous agent, and has many characteristics of clinical X-linked juvenile retinoschisis in human.

### BRIEF DESCRIPTION OF THE DRAWINGS

FIG. 1. Targeted disruption of exon 3 of the Rs1h gene.

(a) In the targeting construct, 1 bp of exon 3 (E3*), all of intron 3, and 66 bp of exon 4 (E4*) are deleted and replaced with a lacZ-neo^{r} cassette. (b) PCR amplification demonstrates the correct targeting of Rs1h. The relative positions of oligonucleotide primers and expected product sizes are given in a. (c) By using a probe spanning exons 4 to 6 of Rs1h, Northern blot analyses reveal the expected 5.6- and 4.9-kb transcripts in eye total RNA from the wt (wt) but not from the Rs1h knock-out (−/Y) male mouse. Subsequent hybridization with a lacZ probe exhibits a fusion transcript of 3.7 kb only in the Rs1h-deficient animal. (d) Western blot analysis using eye cup protein extracts. Polyclonal antibody pAB-ap3RS1 labels the 24-kDa RS1 protein in WT mice. With the same antibody, the expected fusion protein of 120 kDa is not observed in mutant males, although it contains the antibody epitope, thus indicating that the targeted allele represents a true null allele. Equal loading of protein extracts is demonstrated by Coomassic staining.

FIG. 2. Macromorphological evaluation of the Rs1h^{-/Y} retina with scanning laser ophthalmoscopy.

(a) Survey of the fundus, demonstrating a layer of cyst-like elevations in the inner retina. (b) Optical magnification reveals that the densely packed structures are clearly demarcated from the surrounding normal-appearing regions. (c) Focus on the retinal surface shows superficial vessels and the nerve fiber layer. Visible in the lower right quadrant are several larger cysts, one displacing a retinal vessel (arrow). (a) Fundus photograph of a patient with RS, featuring typical small macular cysts arranged in a stellate pattern (arrow) and radial striae centered on the fovea. There is an obvious similarity to the appearance of the mouse retina as shown in b.

FIG. 3. Electrophysiology of Rs1h^{-/Y} and wt mice.

Scotopic intensity series of a wt (a) and an Rs1h mutant mouse (b). Log light intensities (from top to bottom) were −4, −3, −2, −1.5, −1, −0.5, 0, 0.5, 1, 1.5 log cd·s/m². The overall loss of amplitude and the additional selective reduction of the b-wave are clearly visible. Photopic intensity series of a wt control (c) and an Rs1h-deficient mouse (d). Log light intensities (from top to bottom) were −2, −1.5, −1, −0.5, 0, 0.5, 1, 1.5 log cd·/s/m². The photopic ERG of the Rs1h^{-/Y} mice is strongly reduced, indicating a much more severe cone than rod dysfunction.

FIG. 4.(a-d) Semithin retinal sections of wt (WT) and Rs1h^{-/Y} (−/Y) mice at 2 months of age.

In meridional sections, the thickness of the central retina is markedly reduced in Rs1h^{-/Y} mice (b and c) as compared with wt mice (a). In some Rs1h^{-/Y} eyes, photoreceptor outer segments (POS) are present (b), whereas others show partial or complete absence of the POS (c). In Rs1h^{-/Y} eyes with areas of preserved POS, large gaps are present between the cells of the INL (arrows in b). Such gaps are absent in areas with complete degeneration of POS (c). (a) Oblique tangential section through the INL of an Rs1h^{-/Y} retina reveals large extracellular gaps (white arrows). In some of the gaps, cell bodies of microglia are observed (black arrow). os, photoreceptor outer segments; onl, outer nuclear layer; opl, outer plexiform layer, inl, inner nuclear layer; ipl, inner plexiform layer; gcl, ganglion cell layer. [Bars=25 μm (a-c) and 2.8 μm (d).]

FIG. 5.(a-f) Electron microscopy of the retina of wt (WT) and Rs1h^{-/Y} (−/Y) mice at 2 months of age.

(a) In the retina of wt mice, typical ribbon synapses are present at the photoreceptor terminals (black arrows). (b) In the retina of Rs1h^{-/Y} mice, increased extracellular spaces (open arrow) are observed in regions of ribbon synapses (solid arrow). Larger extracellular gaps are present between individual photoreceptor terminals (asterisk). (c) The extracellular gaps (asterisk) in the INL of Rs1h^{-/Y} mice are filled with cellular debris (solid arrows) and membranous whorls. (open arrow). (d) Part of the extracellular debris in the 1N gaps (asterisk) of Rs1h^{-/Y} mice consists of fragmented nerve cell terminals (solid arrow) containing synaptic vesicles. (e) Cells with ultrastructural characteristics of microglia in the retina of Rs1h^{-/Y} mice. In the increased extracellular spaces, cells with long cytoplasmic processes (arrows) are observed. (f) Upon higher magnification, multiple clear vesicles (solid arrow) and electron-dense phagolysosomes (open arrow) are observed in the cytoplasm of the cells. [Bars=0.53 μm (a, b, d, and f) and 1.4 μm (c and e).]

FIG. 6. Immunofluorescence microscopy of retinal cryosections from 2-month-old Rs1h^{-/Y} and wt mice.

(a and b) Rs1 labeling with the Rs1 3R10 monoclonal antibody (red). Image is merged with DAPI nuclear staining (blue) and differential interference contrast (DIC) microscopy. (c and d) Rhodopsin staining with the Rho 1D4 monoclonal antibody. (e and f) Cone opsin labeling with a mixture of polyclonal antibody JH 455 and JH 492. (Insets) Bar=10 μm. (g and h) PAN-SAP antibody labeling of PSD-95 in the OPL and IPL in the wt mouse compared with the TS and OPL in the Rs1h^{-/Y} mouse. Image is merged with DIC image showing the retinal layers. (i and j) Labeling of bipolar cells with the monoclonal antibody Mab 115A10. (k and l) Labeling of Mueller cells and retinal pigment epithelial (RPE) cells with an anti-CRALBP antibody. Abbreviations used are as in FIG. 4, plus (is), inner segment.

FIG. 6. Temporal expression of Rs1h during retinal development in the mouse (from postnatal day P0 to P21).

RT-PCR analysis of Rs1h was done with RS specific primers. The retina-specific cone-rod homeo box-containing gene (CRX) and β-actin were used as control reactions to test for cDNA integrity at all stages of development tested. The figure shows strong expression of the RS1 gene already a day 5, and even at day 3 a considerable amount of RS1 specific nucleic acids can be detected.

### DETAILED DESCRIPTION OF THE INVENTION

It is a discovery of the present inventors that transgenic animals can be made which develop many characteristics of clinical X-linked juvenile retinoschisis in human. These animals provide a useful model for studying retinal diseases, as well as for identifying useful regimens for treating or preventing the diseases. To gain further insight into the functional role of retinoschisin in the sensory neuroretina, the present invention provides a knock-out mouse deficient in Rs1h, the murine ortholog of the human RS1 gene (Gehrig et al. 1999; see Example 1). The retinoschisin-deficient mouse shares several important features with X-linked juvenile retinoschisis and establishes this mouse line as a valuable model for the human condition. The major pathology in the retina of the retinoschisin-deficient mouse appears to be a generalized disruption of cell layer architecture, most evident in the loss of integrity of the inner nuclear layer (formation of cyst-like gaps) and an irregular displacement of cells in various retinal layers. Functionally, electroretinogram (ERG) recordings point to severe impairment of bipolar cell associated pathways and a loss of photoreceptors that is more pronounced in cones than in rods. These features make the Rs1h knock-out mouse an ideal model system e.g. for investigating the feasibility of therapeutic gene and/or protein replacement with the aim to correct the murine RS phenotype. Such studies provide the essential groundwork to ultimately venture gene and/or protein-based therapy for X-linked juvenile retinoschisis in human from the laboratory to the clinic setting.

Accordingly, in one aspect the present invention relates to a transgenic non-human animal comprising a recombinant nucleic acid molecule the presence of which leads to inactivation of the expression of a gene orthologous to the human RS1 gene, and wherein said animal displays one or more clinical symptoms of X-linked juvenile retinoschisis (RS).

In 1997, the RS1 gene causing X-linked juvenile retinoschisis was identified by positional cloning within chromosomal band Xp22.2 and shown to consist of six exons encoding a putative 224-amino-acid protein including a 23-amino-acid hydrophobic signal sequence characteristic of proteins destined for cellular secretion (Sauer et al. 1997). The RS protein, termed retinoschisin, is almost exclusively composed of a discoidin-like domain that is present in many other secreted or membrane-bound proteins implicated in cell adhesion or cell-cell interactions (Baumgartner et al. 1998). In its monomeric reduced form retinoschisin migrates as a 24 kDa polypeptide, but under physiological conditions appears to be secreted as a high-molecular weight protein complex of more than 95 kDa (Grayson et al. 2000, Molday et al. 2001). It is present at the cell surfaces of the inner segments of the rod and cone photoreceptors and in smaller amounts at the membranes of bipolar cells and within the synaptic regions of the inner (IPL) and outer plexiform layers (OPL) (Molday et al. 2001).

The animal model of the present invention is particularly useful, since for the first time the function of the RS1 gene and gene product, respectively, which are etiologically related with the clinical phenotype of the X-linked juvenile retinoschisis could be established in early stages of the development of the disease. Without intending to be bound by theory, it is believed that in early stages of development the aberrant function of retinoschisin determines further progression of the disease. Thus, knowledge of the onset of the disease, i.e. the time the aberrant function or loss of function of the RS1 gene product determines the development of the disease can provide an appropriate time period for the pharmaceutical but also surgical intervention, where the aberrant function of the gene or gene product may be compensated or prevented. This also means that therapeutic intervention is sufficient and only necessary at specific and limited times so that side effects for the patient can be minimized.

It is the object of the present invention to provide such therapeutic regimen, which allow the selective prevention and treatment of eye diseases, in particular retinoschisis. The transgenic non-human animals of the present invention display several important clinical symptoms of human: X-linked juvenile retinoschisis and are therefore appropriate as model system for the human diseases.

Animals according to the present invention include without limitation rodents, such as rats and mice, dogs, cats, pigs, guinea pigs, gerbils, sheep, cows, goats, and horses and rabbits. Transgenic animals are those which have incorporated a foreign gene into their genome. A transgene is a foreign gene or recombinant nucleic acid construct which has been incorporated into a transgenic animal. The transgene may be a wild-type or mutant gene, or one which has been altered to express in an aberrant pattern.

Briefly, transgenic animals are made by injecting egg cells with a nucleic acid construct according to the present, invention. The injected egg cells are then implanted into the uterus of a female for normal fetal development. Animals which develop which carry the transgene are then backcrossed to create heterozygotes for the transgene. Methods for making transgenic animals are well known in the art. See, e.g., Watson, J. D., et al., “The Introduction of Foreign Genes Into Mice,” in Recombinant DNA, 2d Ed., W. H. Freeman & Co., New York (1992), pp. 255-272; Gordon, J. W., Intl. Rev. Cytol. 115:171-229 (1989); Jaenisch, R., Science 240: 1468-1474 (1989); Rossant, J., Neuron 2: 323-334 (1990).

The recombinant DNA molecules of the invention may be introduced into the genome of mammals using any method for generating transgenic animals known in the art. Embryonal target cells at various developmental stages are used to introduce the transgenes of the invention. Different methods are used depending on the stage of development of the embryonal target cell(s). These include, without limitation: 1. Microinjection of zygotes; Brinster, et ai., Proc. Natl. Acad. Sci. (USA) 82: 4438-4442 (1985); 2. Viral integration; Jaenich, R, Proc. Natl. Sci. (USA) 73: 1260-1264; Jahner, et al., Proc. Natl. Acad. Sci. (USA) 82: 6927-6931 (1985); Van der Putten, et al., Proc. Natl. Acad. Sci. (USA) 82: 6148-6152 (1985); 3. Embryonal stem (ES) cells obtained from pre-implantation embryos that are cultured in vitro. Evans, M J., et al., Nature 292: 154-156 (1981), Bradley, M. O., et al., Nature 309: 255-258 (1984); Gossler, et al., Proc. Natl. Acad. Sci. (USA) 83:9065-9069 (1986); Robertson et al., Nature 322: 445448 (1986). Furthermore, methods for producing transgenic animals are generally described by Wagner and Hoppe (U.S. Pat. No. 4,873,191; which is incorporated herein by reference) in “Manipulating the Mouse Embryo; A Laboratory Manual” 2nd edition (eds., Hogan, Beddington, Costantimi and Long, Cold Spring Harbor Laboratory Press, 1994; which is incorporated herein by reference in its entirety). Effective generation of transgenic pigs and mice are also described; see Chang et al., BMC Biotechnol. 2 (1):5 (2002). Generation of transgenic rabbits is described in James et al., J. Mol. Cell Cardiol. 34 (2002), 873-882 and Murakani et al., Theriogenology 57 (2002), 2237-2245. Furthermore, the generation of transgenic sheep is described for example in Kadokawa et al., Domest. Anim. Endocrinol. 24 (2003), 219-229 and Campbell, Methods Mol. Biol. 180 (2002), 289-301. U.S. Pat. No. 5,639,457 is also incorporated herein by reference to supplement the present teaching regarding transgenic pig and rabbit production. U.S. Pat. Nos. 5,175,384; 5,175,385; 5,530,179, 5,625,125, 5,612,486 and 5,565,186 are also each incorporated herein by reference to similarly supplement the present teaching regarding transgenic mouse and rat production.

In one particular method, production of transgenic non-human animal displaying one or more clinical symptoms of X-linked juvenile retinoschisis (RS) comprises the following steps:

(a) introducing a nucleic acid construct comprising at least part of the RS1 gene interrupted in frame by a nucleic acid sequence encoding a reporter gene and a selectable marker gene into an embryo of a non-human animal;

(b) implanting the embryo into a female foster animal of the same species and allow it to develop normally until birth;

(c) screening the offsprings for presence of the nucleic acid construct in the germline; and optionally

(d) mating those offsprings whose germline contains the nucleic acid construct For experimental details see Example 1 and the references cited above. In one embodiment of the method said nucleic acid construct is introduced into an ES cell, screened for the correct integration locus within the RS1 gene and is then transferred into said embryo preferably by microinjection. In another embodiment of the method said nucleic acid is introduced into a fertilized egg of said animal, preferably by microinjection and allowing the egg to divide into an early embryo, which is the transferred into said foster animal. For those embodiments see also methods for generation of transgenic animals described in US 2002/088017 which is incorporated herein by reference in its entirety.

In a preferred embodiment of the present invention, the animals ortholog RS1 gene has been inactivated. The nucleotide and amino acid sequences of the human RS1 gene and the mouse homolog are described in Gehrig et al., 1999; see also Genebank by accession IDs: AF014459, HSXLRSONE1, HSXLRSONE2, HSXLRSONE3, HSXLRSONE4, HSXLRSONE5, HSXLRSONE6 and NM_{—}011302, respectively. Furthermore, the X-linked juvenile retinoschisis precursor protein (XLRS1) encoding gene of Fugu has been described by Brunner et al. (Genome Res. 9 (1999), 437-448); see also Genbank accession nos. AF146687, AF094327 and AAD28797.1. In addition, cDNA fragments isolated from Zebrafish and chicken, respectively, can be obtained from Genbank. They can be used to either clone the full-length cDNA or a genomic. DNA, or they may be used for generation of, for example, a knock-out animal.

The RS1 coding sequence which is used may be derived from any species, including but not limited to human and mouse RS1. The RS1 encoding polynucleotide may, for example, be obtained from rats, mice, dogs, cats, pigs, sheep, cows, goats, horses, and rabbits. Wild-type or mutant, whether naturally-occurring or synthetic, may be used. The polynucleotide may encode a signal sequence or it may have the signal sequence deleted. RS1 protein and corresponding encoding DNA can be prepared according methods well known in the art; see also the references cited herein, for example, Sambrook, J., Maniatis, T., Fritsch, E. F. in Molecular Cloning: A Laboratory Manual, Second Edition, Cold Spring Harbor Laboratory Press, Cold Spring Harbor, NY. 1989.

Hence, in a preferred embodiment the transgenic animal of the invention is a RS1 knock-out animal. The term “knock-out” refers to a partial or complete suppression of the expression of at least a portion of a protein, encoded by an endogenous DNA sequence in a cell. The term “knock-out construct” refers to a nucleic acid sequence that is designed to decrease or suppress expression of a protein encoded by endogenous DNA sequences in a cell. The nucleic acid sequence used as the knock-out construct is typically comprised of: (1) DNA from some portion of the gene (exon sequence, intron sequence, and/or promoter sequence) to be suppressed, and (2) a marker sequence used to detect the presence of the knock-out construct in the cell. The knock-out construct is inserted into a cell, and integrates with the genomic DNA of the cell in such a position so as to prevent or interrupt transcription of the native DNA sequence. Such insertion usually occurs by homologous recombination (i.e., regions of the knock-out construct that are homologous to endogenous DNA sequences hybridize to each other when the knock-out construct is inserted into the cell and recombine so that the knock-out construct is incorporated into the corresponding position of the endogenous DNA). The knock-out construct nucleic acid sequence may comprise 1) a full or partial sequence of one or more exons and/or introns of the gene to be suppressed, 2) a full or partial promoter sequence of the gene to be suppressed, or 3) combinations thereof. Typically, the knock-out construct is inserted into an embryonic stem cell (ES cell) and is integrated into the ES cell genomic DNA, usually by the process of homologous recombination. This ES cell is then injected into, and integrates with, the developing embryo.

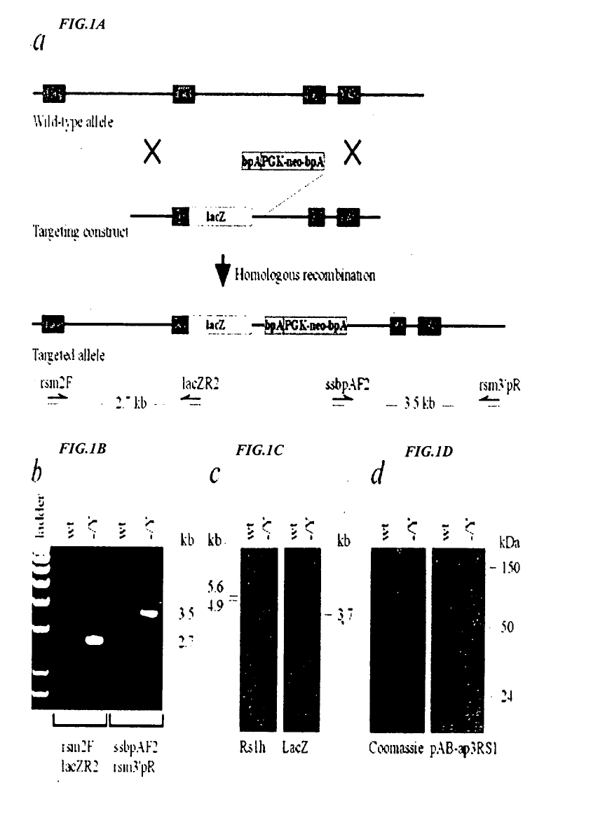

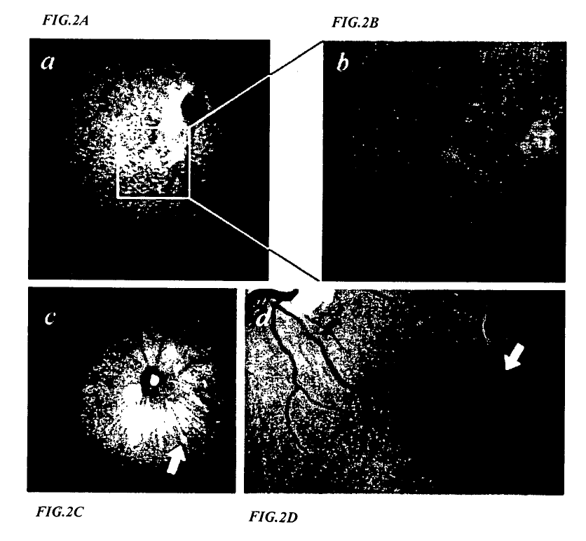

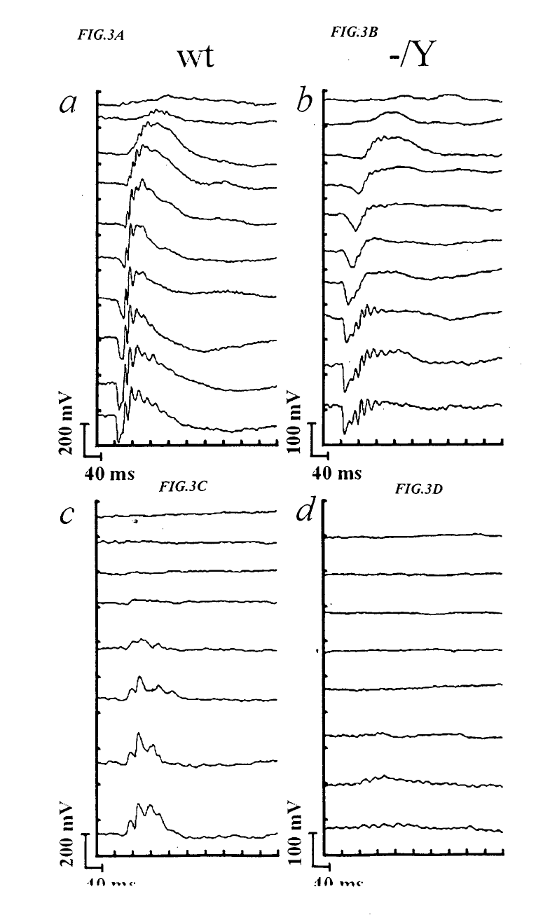

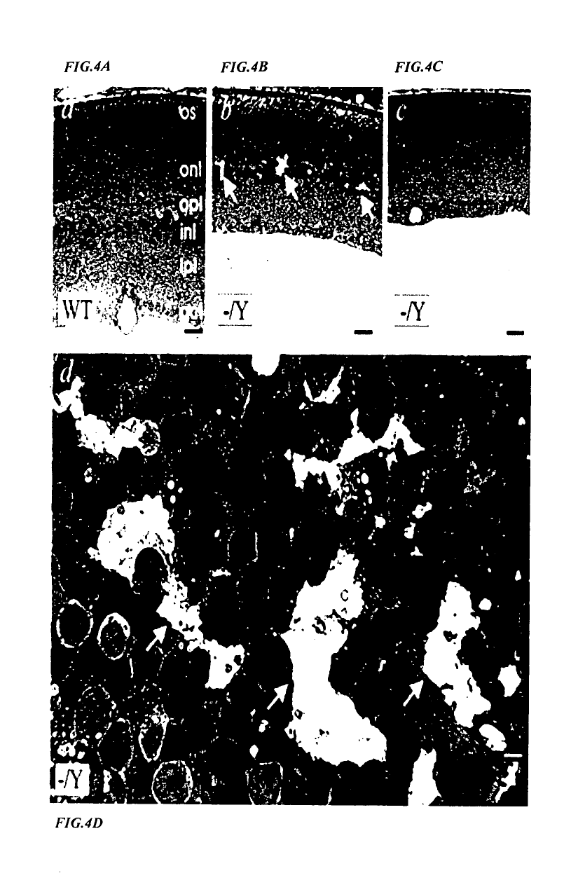

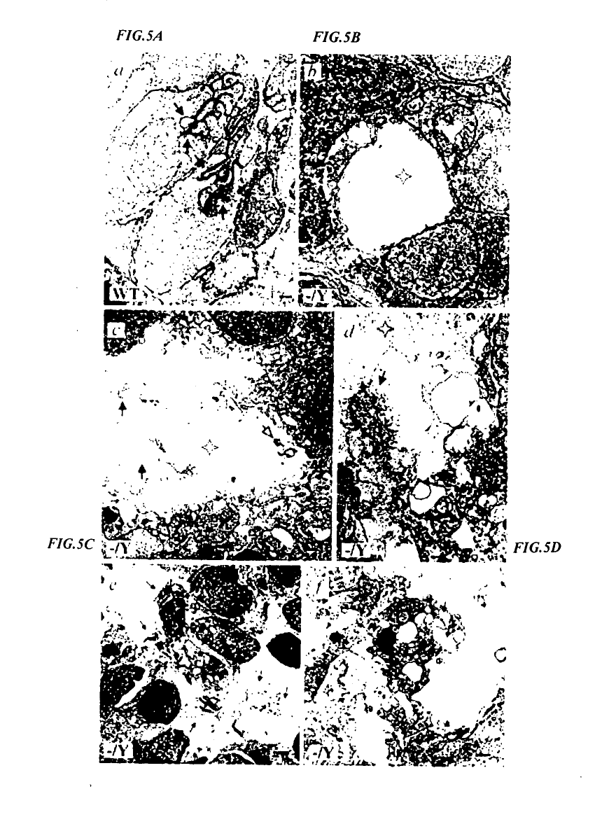

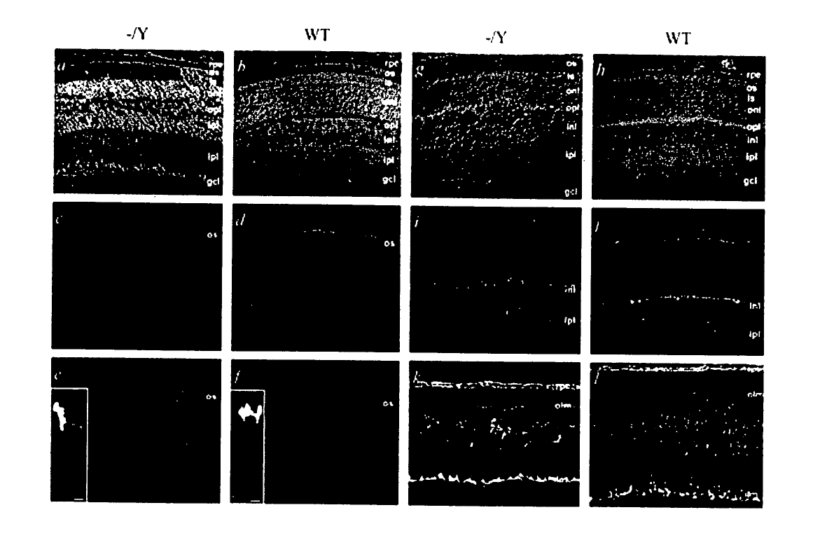

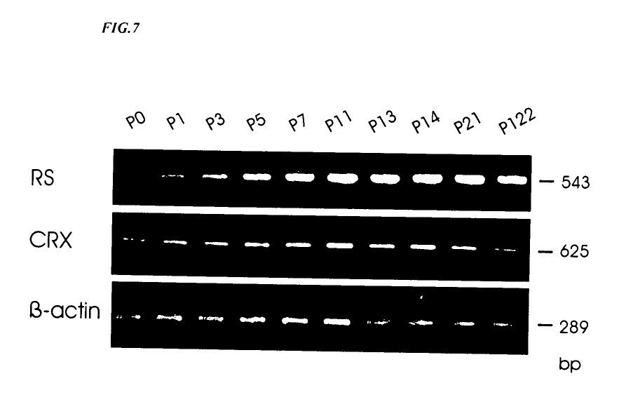

In a preferred embodiment, the transgenic animal has the endogenous RS1 gene disrupted by a polynucleotide encoding a fragment of the RS1 gene in combination with a selection marker; see also Example 1 and FIG. 1. The phrases “disruption of the gene” and “gene disruption” refer to insertion of a nucleic acid sequence into one region of the native DNA sequence (usually one or more exons) and/or the promoter region of a gene so as to decrease or prevent expression of that gene in the cell as compared to the wild-type or naturally occurring sequence of the gene. By way of example, a nucleic acid construct can be prepared containing a DNA sequence encoding an antibiotic resistance gene which is inserted into the DNA sequence that is complementary to the DNA sequence (promoter and/or coding region) to be disrupted. When this nucleic acid construct is then transfected into a cell, the construct will integrate into the genomic DNA. Thus, many progeny of the cell will no longer express the gene at least in some cells, or will express it at a decreased level, as the DNA is now disrupted by the antibiotic resistance gene. Usually, the DNA to be used in the knock-out construct will be one or more exon and/or intron regions, and/or a promoter region from the genomic sequence provided herein, but may also be cDNA sequence.

As described in the examples said polynucleotide preferably encodes a reporter gene and a selectable marker gene flanked by genomic regions of the RS1 gene within the same open reading frame. The marker gene can be any nucleic acid sequence that is detectable and/or assayable, as for example an antibiotic resistance gene such as neo (the neomycin resistance gene) or a gene, such as beta-galactosidase; however typically it is an antibiotic resistant gene or other gene whose expression or presence in the genome can easily be detected. In a preferred embodiment, the marker gene in the neomycin resistance gene. The marker gene is usually operably linked to its own promoter or to another strong promoter from any source that will be active or can easily be activated in the cell into which it is inserted; however, the marker gene need not have its own promoter attached as it may be transcribed using the promoter of the RS1 gene to be suppressed. In addition, the marker gene will normally have a polyA sequence attached to the 3′ end of the gene; this sequence serves to terminate transcription of the gene.

After the genomic DNA sequence has been digested with the appropriate restriction enzymes, the marker gene sequence is ligated into the genomic DNA sequence using methods well known to the skilled artisan (for example Sambrook et al., 1989). The ends of the DNA fragments to be ligated must be compatible; this is achieved by either cutting all fragments with enzymes that generate compatible ends, or by blunting the ends prior to ligation. Blunting is done using methods well known in the art, such as, for example, by the use of Klenow, fragment (DNA polymerase I) to fill in sticky ends. The ligated knock-out construct may then be inserted directly into embryonic stem cells.

Preferably, the reporter gene is the LacZ gene and the selectable marker gene confers an antibiotic resistance, preferably a neomycine resistance. In a particularly preferred embodiment, the DNA to be used in the knock-out construct comprise exon 3 upstream and intron 3 plus exon 4 downstream of the reporter and marker gene, respectively; see also Example 1. Generally, the DNA will be at least about 500 bp to 1 kilobase (kb) in length, and in certain aspects up to, 3-4 kb in length, thereby providing sufficient complementary sequence for hybridization when the knock-out construct is introduced into the genomic DNA of the ES cell.

In certain embodiments of the present invention, rescue of a RS1 gene or genetic construct may be desired. The present invention contemplates the use of site-specific recombination systems to rescue specific genes out of a genome, and to excise specific transgenic constructs from the genome. Members of the integrase family are proteins that bind to a DNA recognition sequence, and are involved in DNA recognition, synapsis, cleavage, strand exchange, and religation. Currently, the family of integrases includes 28 proteins from bacteria, phage, and yeast which have a common invariant His-Arg-Tyr triad (Abremski and Hoess, Protein Eng. 5 (1992), 87-91). Among of the most widely used site-specific recombination systems for eukaryotic applications The Cre-loxP and FLP-FRT systems have been developed to the greatest extent. The R-RS system, like the Cre-loxP and FLP-FRT systems, requires only the protein and its recognition site. The Gin recombinase selectively mediates DNA inversion between two inversely oriented recombination sites (gix) and requires the assistance of three additional factors: negative supercoiling, an enhancer sequence and its binding protein Fis. The present invention contemplates the use of the Cre/Lox site-specific recombination system (Sauer, Methods in Enzymology, 225 (1993), 890-900, available through Gibco/BRL, Inc., Gaithersburg, Md.) to rescue specific genes out of a genome, and to excise specific transgenic constructs from the genome.

Preferably, the transgenic animal of the present invention is a mammal, most preferably a rodent and particularly preferred a mouse. However, the widely used zebra fish may also be used since this model system has also been shown to provide valuable predictive results; see, e.g. Gerlai et al., Pharmacol. Biochem. Behav. 67 (2000), 773-782; see also supra.

As described in the examples the transgenic animal of the present invention displays one or more of the clinical symptoms of retinoschisis; see also example 1. Preferably, the symptoms reflect those of macular degeneration. Hence, the transgenic animals of the present invention preferably develope small cyst-like structures in the inner retina. In addition or alternatively in the analysis of the transgenic animal of the present invention the dark-adapted ERG measurements show a dramatic loss of the positive b-wave when compared to control animals and the light adapted ERG responses are virtually absent. Likewise preferred is that the rod function is not impaired and/or that the retinal layers are disorganized.

In another aspect, the present invention relates to a method of producing a non-human animal displaying one or more clinical symptoms of X-linked juvenile retinoschisis (RS) comprising introducing one or more nucleic acid molecules comprising a nucleotide sequence derived from the human RS1 gene or from a corresponding ortholog or a vector encoding and capable of expressing such nucleic acid molecules into a cell or tissue of the animal, wherein said nucleic acid molecule is capable of provoking the degradation of the corresponding mRNA encoding RS1 or an orthologous gene product.

Such nucleic acid molecules include, for example, a ribozyme, antisense or sense nucleic acid molecules to said RS1 gene or dsRNA molecules which are capable of mediating RNA interference. Methods and computer programs for the preparation rational selection of for example antisense oligonucleotide sequences are described in the prior art; see for example Smith, Eur. J. Pharm. Sci. 11 (2000), 191-198; Toschi, Methods 22 (2000), 261-269; Sohail, Adv. Drug Deliv. Rev. 44 (2000), 23-34; Moulton, 3. Comput. Biol. 7 (2000), 277-292. These procedures comprise how to find optimal hybridization sites, and secondly on how to select sequences that bind to for example mRNA of the RS1 gene. These methods can include the more empirical testing of large numbers of mRNA complementary sequences to the more systematic techniques, i.e. RNase H mapping, use of combinatorial arrays and prediction of secondary structure of mRNA by computational methods. Structures that bind to structured RNA, i.e. aptastructures and tethered oligonucleotide probes, and foldback triplex-forming oligonucleotides can also be employed for the purpose of the present invention. Secondary structure prediction and in vitro accessibility of mRNA as tools in the selection of target sites is described for example in Amarzguioui, Nucleic Acids Res. 28 (2000), 4113-4124. Minimising the secondary structure of DNA targets by incorporation of a modified deoxynucleoside: implications for nucleic acid analysis by hybridisation is described in Nguyen, Nucleic Acids Res. 28 (2000), 3904-3909.

Relating to selection of antisense sequences by aid of computational analysis, valuable www addresses are given below.

In a particularly preferred embodiment of the present invention said nucleic acid molecule substantially consists of ribonucleotides which preferbly contain a portion of double-stranded oligoribonucleotides (dsRNA). Desirably, the region of the double stranded RNA that is present in a double stranded conformation includes at least 5, 10, 20, 30, 50, 75, 100 or 200. Preferably, the double stranded region includes between 15 and 30 nucleotides, most preferably 20 to 25 and particularly preferred 21 to 23 nucleotides, since for the specific inhibition of a target gene, it suffices that a double-stranded oligoribonucleotide exhibits a sequence of 21 to 23 nucleotides (base pairs) in length identical to the target gene; see, e.g., Elbashir et al., Methods 26 (2002), 199-213 and Martinez et al., Cell 110 (2002), 563-574. General means and methods for cell based assays for for identifying nucleic acid sequences that modulate the function of a cell, by the use of post-transcriptional gene silencing including definitions, methods for the preparation of dsRNA, vectors, selectable markers, compositions, detection means, etc., and which can be adapted in accordance with the teaching of the present invention are described in European patent application EP 1 229 134 A2, the disclosure content of which is incorporated herein by reference

dsRNA between 21 and 23 nucleotides in length is preferred. The dsRNA molecule can also contain a terminal 3′-hydroxyl group and may represent an analogue of naturally occurring RNA, differing from the nucleotide sequence of said gene or gene product by addition, deletion, substitution or modification of one or more nucleotides. General processes of introducing an RNA into a living cell to inhibit gene expression of a target gene in that cell comprising RNA with double-stranded structure, i.e. dsRNA or RNAi are known to the person skilled in the art and are described, for in WO99/32619, WO01/68836, WO01/77350, WO00/44895, WO 01/75164, WO02/055692 and WO02/055693, the disclosure content of which is hereby incorporated by reference.

The target mRNA of said dsRNA is preferably encoded by an RS1 gene or a cDNA obtained described above. In Examples 5 to 7, Rs1h knock-down by post transcriptional gene silencing via application of Rsh1-specific dsRNA in wt C57BL/6 mice is described.

Vectors that can be used for the purposes in accordance with the teaching of the present invention are known to the person skilled in the art; see, e.g., heritable and inducible genetic interference by double-stranded RNA encoded by transgenes described in Tavernarakis et al., Nat. Genet. 24 (2000), 180-183. Further vectors and methods for gene tranfer and generation of transgenic animals are described in the prior art; see, e.g., adeno-associated virus related vectors described in Qing et al., Virol. 77 (2003), 2741-2746; human immunodeficiency virus type 2 (HIV-2) vector-mediated in vivo gene transfer into adult rabbit retina described in Cheng et al. Curr. Eye Res. 24 (2002), 196-201, long-term transgene expression in the RPE after gene transfer with a high-capacity adenoviral vector described in Kreppel et al., Invest. Ophthalmol. Vis. Sci. 43 (2002), 1965-1970 and non-invasive observation of repeated adenoviral GFP gene delivery to the anterior segment of the monkey eye in vivo described in Borras et al., J. Gene Med. 3 (2001), 437-449.

The expression need not be exclusively in the retina. For example, promoters which are activated in the RPE as compared to other tissues may be used, even though their expression is not solely found in the retina. Suitable promoters for use in the present invention include the rhodopsin promoter, the opsin promoter, the 1RBP promoter, neuron specific enolase promoter, the tyrosinase-related protein-1 promoter, the angiopoietin 2 promoter; see, e.g., Raymond, Current Biology, 5 (1995), 1286-1295; Lowings, Mol. Cell Biology 12 (1992), 3653-3662, Jackson, Nucleic Acids Research 19 (1991), 3799-3804; Beermann, Cell Mol. Biol. 45 (1999), 961-968; Hackett, J. Cell. Physiol. 184 (2000), 275-284. Other suitable promoters can be found by looking for differentially displayed genes in libraries of retinally expressed or retinal pigmented epithelium-expressed genes. particularly preferred to promoters directing the expression in the cells and tissue of the eye.

The present invention also relates to the animal obtainable by the method described above and which displays, due to the presence of the nucleic acid molecule defined herein before, on or more clinical symptoms of retinoschisis as mentioned before and described in the examples. Preferably, said animal is a mouse.

In addition, the present inveniton relates to polynucleotides and nucleic acid molecules as defined herein-before that can be used for producing a transgenic animal of the present invention; see supra.

The transgenic animals of the present invention can be used to screen regimens for prevention and treatment of retinoschisis. Thus if regimens are provided before the disease develops or the onset takes place, such as is before postal day 5 in mice, or even before postal day 4 or 3; see FIG. 7 which shows the expression of RS1, and retinoschisis is delayed or prevented, then a prophylactic regimen has been identified. If the regimen is administered after retinoschisis has developed, and the regimen causes a reduction, cessation, or regression, then a therapeutic regimen has been identified. The regimen may be administration of a test compound or other medicinal chemistry or natural products sample. The regimen may also be application of a dye and laser or administration of a foreign gene. Even surgical techniques can be tested on the transgenic animal model of the present invention.

Thus, in a further aspect the present invention relates to a method of screening test therapies as potential prevention or treatment of retinoschisis comprising determining the time frame for the onset or development of one or more of the clinical symptoms displayed by any one of the transgenic non-human animals of the invention described herein-before. Said symptoms can be determined, for example, by Scanning-Laser Ophthalmoscopy, Electroretinogram, Histology and Electron Microscopy, Immunofluorescence Labeling, and/or Cone Photoreceptor Count; see also the method section in the appended examples.

In a preferred embodiment of the invention, the method further comprises the steps of:

(a) administering a composition comprising a test compound known to be capable to compensate for the loss of expression of the RS1 gene or for the activity of the RS1 gene product to the animal at different times and/or dosages within one of the identified time frames;

(b) monitoring said animal for alleviating the symptoms; and

(c) determine the optimal administration time, release and/or dosage regimen.

The test compound can be for example a functional RS1 protein; see also Examples 2 to 4. Furthermore, the test compound may be formulated in a composition for retarded release and/or release at predetermined time after administration of the composition. This embodiment is particularly useful for making pharmaceutical compositions as effective as possible for the treatment of the disease.

As described in examples, compensation of the aberrant or loss of function of the RS1 gene can be accomplished in accordance with the present invention by adeno-associated viral based gene transfer mediated introduction of a functional form of the RS1-gene and gene product, respectively. On the other hand, local application of peptidic substances can be performed such as wild type RS1 protein or a recombinant form thereof, which take over the function of the endogenous protein in the specific metabolic context. The mentioned animal models provide the possibility to validate the described methods and treatment by the specific early intervention in respect to their protective effect on the onset of X-linked juvenile retinoschisis. Hence, in a preferred embodiment of the invention said test therapy is a gene therapy or a protein replacement therapy. Both of these therapies are described in the examples. Functional and structural recovery of the retina after gene therapy in the RPE65 null mutation dog has been described in Narfstrom et al., Invest. Ophthalmol. Vis. Sci. 44 (2003), 1663-1672. Thus, transgenic dogs prepared according to the method of the present invention can also be used for the screening method.

Advantages provided by the present invention inter alia reside in the provision of animal models, which substantially display the clinical appearance of X-linked juvenile retinoschisis and which therefore are suitable for testing medical interventions such as the application of, e.g., peptidic substances or gene transfer vectors, or surgery in the early stages of development of the disease before onset of the clinical symptoms of the disease. Those models provide a prerequisite for therapy and treatment of X-linked juvenile retinoschisis.

Thus, the present invention also relates to a method of screening and/or isolating compounds having therapeutic activity in the treatment of retinal disorders comprising:

(a) administering a test compound to a transgenic non-human animal of the present invention described above; and

(b) monitoring said animal to determine if the compound is alleviating the symptoms.

A suitable drug can be identified by observing whether a candidate compound is able at a certain concentration to prevent or revert one or more of the clinical symptoms of retinoschisis of said transgenic non-human animal back to normal, i.e. wild typ animal. In a particular preferred embodiment, said transgenic non-human animal displays one or more symptoms as defined above. In accordance with the method of the invention for screening test therapies, said test compound is preferably administered in a time, and/or dosage regimen and/or retard formulation determined according to any one of the previous methods.

The test substances which can be tested and identified according to a method of the invention may be expression libraries, e.g., cDNA expression libraries, peptides, proteins, nucleic acids, antibodies, small organic compounds, hormones; peptidomimetics, PNAs, aptamers or the like (Milner, Nature Medicine 1 (1995), 879-880; Hupp, Cell 83 (1995), 237-245; Gibbs, Cell 79 (1994), 193-198 and references cited supra). The test substances to be tested also can be so called “fast seconds” of known drugs. The invention also relates to further contacting the test cells with a second test substance or mixture of test substances in the presence of the first test substance.

The above-described methods can, of course, be combined with one or more steps of any of the above-described screening methods or other screening methods well known in the art. Methods for clinical compound discovery comprises for example ultrahigh-throughput screening (Sundberg, Curr. Opin. Biotechnol. 11 (2000), 47-53) for lead identification, and structure-based drug design (Verlinde and Hol, Structure 2 (1994), 577-587) and combinatorial chemistry (Salemme et al., Structure 15 (1997), 319-324) for lead optimization.

Furthermore, the present invention relates to a pharmaceutical composition comprising a compound identified or isolated according to the above-described method of the present invention, preferably wherein said composition is formulated so as to release the compound at the time and/or in dosage determined according to the method of screening test therapies. General methods for the preparation of retard compositions, i.e compositions for controlled release of drugs are known in the art; see, e.g., Gupta et al., Drug Discov. Today 7 (2002), 569-579. Recent advances in the stabilization of proteins encapsulated in injectable PLGA delivery systems, which according to the present invention may be used for the delivery of functional RS1 protein or corresponding functional analogue, have been described by Vermani and Garg, Crit. Rev. Ther. Drug Carrier Syst. 19 (2002), 73-98.

Preferably, the dosage forms comprise ones which affect the precorneal parameters, and those that provide a controlled and continuous delivery to the pre- and intraocular tissues. The systems the commonly used dosage forms such as gels, viscosity imparting agents, ointments, and aqueous suspensions, newer concept of penetration enhancers, phase transition systems, use of cyclodextrins to increase solubility of various drugs, vesicular systems, and chemical delivery systems such as the prodrugs, the developed and under-development controlled/continuous drug delivery systems including ocular inserts, collagen shields, ocular films, disposable contact lenses, and other new ophthalmic drug delivery systems, and the newer trends directed towards a combination of drug delivery technologies for improving the therapeutic response of a non-efficacious drug. An overview of topical ocular drug delivery systems is given in Kaur and Kanwar, Drug. Dev. Ind. Pharm. 28 (2002), 473-493.

Preparation of pharmaceutical compositions according to the identified therapy regimen and applying the above-mentioned technological suggestions can result in a superior dosage form, especially for both topical and intraocular ophthalmic application, in particular for the treatment of retinoschisis.

The compositions of the invention may be administered locally or systemically e.g., intravenously. Preferably, the compositions are administered as eye drops or systemically, iontophoretically or by retrobulbar injection.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. Furthermore, the pharmaceutical composition of the invention may comprise further agents such as interleukins or interferons depending on the intended use of the pharmaceutical composition.

In accordance with the present invention the pharmaceutical compositions are administered to a subject in an effective dose of between about 0.1 μg to about 10 mg units/day and/or units/kg body weight.

Furthermore, the present invention relates to the use of a compound identified, isolated and/or produced by any of these methods for the preparation of a composition for the treatment of retinoschisis. As a method for prevention or treatment of retinoschisis comprising administering to a subject in need thereof a therapeutically effective amount of a compound capable to compensate for the loss of expression of the RS1 gene or for the activity of the RS1 gene product to the subject. Preferably, said compound is administered in a tine and/or dosage regimen determined according to the method of the invention described above.

In one embodiment of the present invention, said compound to be administered is native wild type RS1 protein or a recombinant RS1 protein, or functional derivative or analogue thereof. Administration of recombinant and not recombinant retinoschisin protein Rs1h for supplementation in Rs1 h-deficient mice (Rs1h^{-/Y}) is described in Examples 2 to 4.

Preferably, the recombinant RS1 protein or functional derivative or analogue thereof is not larger than the “bioavailability wall” of 500-600 Da in order to be able to cross the lipophilic cell membrane into the cell. On the other hand, in protein therapy it has been recently demonstrated that enzymes fused to part of a protein from the HIV virus can cross cell membranes while retaining their enzymatic activity in vivo in mice (Schwarze, Science 285 (1999), 1569-1572). It has been known for approximately ten years that the transactivating regulatory protein (TAT protein) from the HIV virus has an unusual ability to cross cell membranes without using receptors or transporters, or requiring ATP (Green and Loewenstein, Cell 55 (1988), 1179-1188). Although its exact mechanism is unknown, it has been shown that the protein transduction domain (PTD) of TAT opens a “hole” in the cell membrane lipid bilayer, pulling anything covalently attached through it, before closing it again. This is a specific process that does not otherwise damage the cell. Thus, a recombinant RS1 protein or functional derivative or analogue thereof may be coupled to PTD via a linker in order to let them cross the cell membrane; see also for review DDT 4 (1999), 537.

Gene therapy, which is based on introducing therapeutic genes into cells by ex-vivo or in-vivo techniques is one of the most important applications of gene transfer. Transgenic mice expressing a neutralizing antibody directed against nerve growth factor have been generated using the “neuroantibody” technique; Capsoni, Proc. Natl. Acad. Sci. USA 97 (2000), 6826-6831 and Biocca, Embo J. 9 (1990), 101-108. Suitable vectors, methods or gene-delivering systems for in-vitro or in-vivo gene therapy are described in the literature and are known to the person skilled in the art; see, e.g., Giordano, Nature Medicine 2 (1996), 534-539; Schaper, Circ. Res. 79 (1996), 911-919; Anderson, Science 256 (1992), 808-813, Isner, Lancet 348 (1996), 370-374; Muhlhauser, Circ. Res. 77 (1995), 1077-1086; Onodua, Blood 91 (1998), 30-36; Verzeletti, Hum. Gene Ther. 9 (1998), 2243-2251; Verma, Nature 389 (1997), 239-242; Anderson, Nature 392 (Supp. 1998), 25-30; Wang, Gene Therapy 4 (1997), 393-400; Wang, Nature Medicine 2 (1996), 714-716; WO 94/29469; WO 97/00957; U.S. Pat. No. 5,580,859; U.S. Pat. No. 5,589,466; U.S. Pat. No. 4,394,448 or Schaper, Current Opinion in Biotechnology 7 (1996), 635-640, and references cited therein. In particular, said vectors and/or gene delivery systems are also described in gene therapy approaches in neurological tissue/cells (see, inter alia Blömer, J. Virology 71 (1997) 6641-6649) or in the hypothalamus (see, inter alia, Geddes, Front Neuroendocrinol. 20 (1999), 296-316 or Geddes, Nat. Med. 3 (1997), 1402-1404). Further suitable gene therapy constructs for use in neurological cells/tissues are known in the art, for example in Meier (1999), J. Neuropathol. Exp. Neurol. 58, 1099-1110. The nucleic acid molecules and vectors of the invention may be designed for direct introduction or for introduction via liposomes, viral vectors (e.g. adenoviral, retroviral), electroporation, ballistic (e.g. gene gun) or other delivery systems into the cell. The introduction and gene therapeutic approach should, preferably, lead to the expression of a functional copy of the target gene of RS1. In some embodiments, the nucleic acid molecules are perferably linked to cell and/or tissue specific promoters; see supra.

Hence, in a particular preferred embodiment of the present invention, the compound used for prevention or treatment of retinoschisis is a recombinant nucleic acid molecule encoding RS1 protein or a functional derivative or analogue thereof. Preferably, said recombinant nucleic acid molecule is comprised in a gene transfer vector. Gene therapy vectors for genetic and acquired retinal diseases are known in the art; see, e.g., Nickells R, et al., Surv. Ophthalmol. 47 (2002), 449-469; Borras et al., Invest. Ophthalmol. Vis. Sci. 43 (2002), 2513-2518. In particular, ΛΛV-mediated gene transfer is described, for example in Mori, Invest. Ophthalmol. Vis. Sci. 43 (2002), 1994-2000. In a particular preferred embodiment of the present invention, said recombinant nucleic acid molecule is a recombinant adeno-associated virus (rAAV) based gene therapy vector, preferably wherein the expression of said RS1 protein or functional derivative or analogue thereof is under the control of the opsin promoter; see Example 8 and 9. However, other vectors, promoters and methods for gene transfer can be used as well, for example those described above in context with generating a transgenic animal of the present invention.

The dosage regimen of the pharmaceutical compositions in all of the above described methods and uses of the present invention can be further refined by the attending physician according to clinical factors. As is well known in the medical arts, dosages for any one patient depends upon many factors, including the patient's size, body surface area, age, the particular compound to be administered, sex, time and route of administration, general health, and other drugs being administered concurrently. A typical dose can be, for example, in the range of 0.001 μg to 10 mg (or of nucleic acid for expression or for inhibition of expression in this range); however, doses below or above this exemplary range are envisioned, especially considering the aforementioned factors. Generally, the regimen as a regular administration of the pharmaceutical composition should be in the range of 0.01 μg to 10 mg units per day. If the regimen is a continuous infusion, it should also be in the range of 0.01 μg to 10 mg units per kilogram of body weight per minute, respectively. Progress can be monitored by periodic assessment. Dosages will vary but a preferred dosage for intravenous administration of nucleics acids is from approximately 10⁶ to 10¹² copies of the nucleic acid molecule.

A therapeutically effective dose refers to that amount of compounds described in accordance with the present invention needed to ameliorate the symptoms or condition. Therapeutic efficacy and toxicity of such compounds can be determined by standard pharmaceutical procedures in cell cultures or experimental animals, e.g., ED50 (the dose therapeutically effective in 50% of the population) and LD50 (the dose lethal to 50% of the population). The dose ratio between therapeutic and toxic effects is the therapeutic index, and it can be expressed as the ratio, LD50/ED50.

Alternatively, prevention or treatment of retinoschisis in accordance with the present invention comprises surgical intervention in a time frame determined according to the method of screening test therapies described above. Those physical approaches for the treatment include laser photocoagulation, photodynamic therapy (using verteprofin, trade name Visudyne®, Novartis), irradiation and or surgical therapies.

These and other embodiments are disclosed and included in the present description and in the examples. Literature regarding the materials, methods, applications and components, which can be used in accordance with the invention, may be obtained from public libraries and data bases, for example by using electronic devices. The public data base ‘Medline’ may for instance be used, which is supported by the National Center for Biotechnology Information and/or the National Library of Medicine at the National Institutes of Health. Other data bases and Internet addresses, such as the European Bioinformatics Institute (EBI), which is part of the European Molecular Biology Laboratory (EMBL), are known to the person skilled in the art, and can be found by using Internet search engines. A survey of patent information in biotechnology and a summary of relevant sources for patent information, which are useful for a retrospective search and current awareness are described in Berks, TIBTECH 12 (1994), 352-364.

The disclosure above describes the present invention in general. A more comprehensive understanding of the invention may be gained by reference to the following specific examples and figures, which are merely provided for illustrative purposes and are not intended to limit the scope of the invention. The contents of all cited references (including literature references, granted patents, published patent applications as quoted in the text and manufacturer's descriptions and specifications, etc.) are hereby incorporated explicitly by reference; this is however no admission that any one of these documents is indeed prior art as to the present invention.

Unless stated otherwise, the present invention may be carried out by making use of conventional techniques of cell biology, cell culture, molecular biology, transgenetic biology, microbiology, recombinant DNA and RNA technology, which belong to the skill of the person skilled in the art. For a comprehensive description of such techniques in the literature, see for example: Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Harnes & S. J. Higgins eds. 1984); Transcription And Translation (B. D. Harnes & S. J. Higgins eds. 1984); Culture Of Animal Cells (R. I. Freshney, Alan P Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986).

### EXAMPLES

### Example 1

### Generation and Characterization of Rs1h-Deficient Mice

Disruption of the Rs1h gene was obtained by introducing a lacZ reporter gene in-frame into exon 3 of the Rs1h gene together with a neomycin-resistance gene (neo) expression cassette under the separate control of the mouse phosphoglycerate kinase gene (Pgk) promoter (FIG. 1a). Successful germline transmission of the correctly targeted allele was confirmed in the F1 generation by PCR analysis of tail DNA with primers rsm2F/lacZR2 and ssbpAF2/rsm3′pR (FIGS. 1a and b). Carrier female offspring (Rs1h^{-/+}) were mated with C57BL/6 male mice to obtain hemizygous Rs1h^{-/Y} males whose general appearance was indistinguishable from their wt litter mates. Northern blot analysis of eye cup RNA from 6-week-old mice with an Rs1h3′-UTR probe detects 5.6- and 4.9-kb transcripts in wt but not in Rs1h^{-/Y} mice, whereas a lacZ probe reveals the expected 3.7-kb fusion transcript in the mutant animals (FIG. 1c). However, the translation product of the fusion transcript was not detected by Western blot analysis with the pAB-ap3RS1 antibody directed against the N terminus of human retinoschisin (Molday et al. 2001), indicating that the disrupted Rs1h locus represents a true null allele (FIG. 1d).

In vivo imaging of 3-month-old Rs1h^{-/Y} mice with an SLO revealed a densely packed layer of small cyst-like structures in the inner retina, sometimes extending to the nerve fiber layer (FIGS. 2a and b). In contrast to the human condition where the cyst formation is largely restricted to the macular area (FIG. 2d), their distribution was homogenous across the entire retina (FIGS. 2a and b). Similar to human, larger cysts were observed in the retinal periphery of Rs1h^{-/Y}, which, in some instances, displaced superficial retinal vessels (FIG. 2c).

Analogous to the “negative ERG” typically observed in human RS (Kellner et al. (1990) Arch. Clin. Exp. Ophthalmol. 228, 432437; Hirose et al. (1977) Doc. Ophthalmol. Proc. Ser. 13, 173-184), dark-adapted (scotopic) ERGs in the Rs1h^{-/Y} mice showed a dramatic loss of the positive b-wave, which is mostly shaped by the neurons of the inner retina. Although amplitudes were less than one-half of normal, the negative a-wave representing both inner and outer retinal components was relatively preserved (FIGS. 3a and b). Under light-adapted (photopic) conditions, ERG responses were virtually absent, suggesting a profound dysfunction of the cone system (FIGS. 3c and d). A more specific analysis of rod photoreceptor responses using the double flash method (Hetling and Pepperberg (1999) J. Physiol. 516, 593-609) did not show detectable abnormalities beyond amplitude reduction (data not shown), indicating that Rs1h deficiency does not specifically impair rod function. Taken together, the ERG findings suggest a decrease in the number of functional photoreceptors with the remaining cells responding normally to light stimuli. The additional selective attenuation of the b-wave, in conjunction with the retinal depth estimate of the cyst layer by SLO and previous evidence that retinoschisin is associated with photoreceptors and bipolar cells, point to the bipolar cell layer or the bipolar cell/photoreceptor connection as likely sites of pathology in Rs1h^{-/Y} mice.

Histologic examination of retina sections from two-month-old wt and Rs1h^{-/Y} mice showed striking changes in the inner and outer nuclear layers (INL and ONL) of the mutant animals. Essentially, a pronounced disorganization of the retinal layers was observed, accompanied by a significant reduction in the number of photoreceptor nuclei (FIG. 4a-c). In two Rs1h^{-/Y} eyes, areas with preserved photoreceptor outer segments (POS) were still present at this age (FIG. 4b), whereas one mutant animal revealed an almost complete absence of POS over the entire retinal sections (FIG. 4c), suggesting a certain degree of heterogeneity in disease phenotype. Mainly in those areas where photoreceptor outer segments were still present, large schisis-like gaps were observed between the cells of the inner nuclear layer (FIG. 4b). In areas of Rs1h^{-/Y} retinae where complete loss of photoreceptor outer segments was obvious, schisis formation was not evident histologically (FIG. 4c).

Normal ribbon synapses were found by electron microscopy at the photoreceptor terminals of a two-month-old wt mouse (FIG. 5a). In contrast, the retina of an Rs1h^{-/Y} littermate revealed increased extracellular spaces in the region of photoreceptor ribbon synapses. In addition, larger extracellular gaps were present between individual photoreceptor terminals (FIG. 5b) and the perikarya of the inner nuclear layer (FIG. 5c). The extracellular gaps (asterisk in FIG. 5c) in the inner nuclear layer of the Rs1h^{-/Y} retina were filled with membranous whorls and cellular debris containing fragmented mitochondria and nerve-cell terminals (FIG. 5c and a). In addition, cells with ultrastructural characteristics of microglia, which expressed long cytoplasmic processes, multiple clear vesicles, and electron-dense phagolysosomes were present in the spaces (FIGS. 5e and f).

To delineate further the consequences of Rs1h deficiency for specific retinal cell types, cryosections from wt and Rs1h^{-/Y} retinas were labeled with cell-specific antibodies for analysis by immunofluorescence microscopy (FIGS. 6a-l). Supporting our Western blot results (FIG. 1d), Rs1 3R10 antibody labeling was absent from the mutant retina (FIG. 6a). DAPI staining identifies some nuclei (cells) past the outer limiting membrane and in the inner and outer segment layers as well as displacement of nuclei into the inner plexiform layer (IPL; FIG. 6a). The wt retina shows a typical distribution of Rs1h with the protein localizing throughout much of the inner and outer retina with intense staining of the photoreceptor inner segments and bipolar cells (FIG. 6b). in addition, significant amounts of Rs1h are present in the IPL and outer plexiform layer (OPL). Immunolabeling of rhodopsin: shows that it is translocated to the outer segments in the Rs1h^{-/Y} mouse, although the outer segments appear disorganized compared with those in wt retina (FIGS. 6c and d). Analysis of short and medium cone opsin-immunolabeled cells indicated that there were three times fewer cones in the retina Rs1h^{-/Y} mice than in wt mice. A marked delocalization of opsin to the inner segment, cell body, and synaptic region was evident in many cone photoreceptors of the Rs1h^{-/Y} knock-out, but not wt, mice (FIGS. 6e, f, and Insets). Labeling of the postsynaptic density (PSD)-95 MAGUK protein with the PAN-SAP or 7E3-1B8 antibody produces immunoreactivity in the OPL and IPL of the retina of the wt mouse (FIG. 6h), similar to that described for rat retina (Koulen et al. (1998) J. Neurosci. 18, 10136-10149) In the Rh retina, the staining pattern reveals significant disorganization of the OPL and an accumulation of PSD-95 in the photoreceptor inner segment (FIG. 6g). In addition, there is a significant decrease in PSD-95 in the IPL (FIG. 6g). By using monoclonal antibody Mab 115A10, substantial disorganization also is evident that involves the INL bipolar cells of the mutant retina (FIGS. 6i and j). Staining of the Mueller cells with an anti-CRALBP antibody shows a similar pattern in both knock-out and wt mice; however, in the mutant retina, areas devoid of staining within the INL and more intense staining of the inner limiting membrane were observed (FIG. 6 k and l).

### Example 2

### Intrabulbar Application of Recombinant or not Recombinant Retinoschisin Protein Rs1h (A Polypeptide of 24 kD) in the Rs1h^{-/Y} Mouse Animal Model Described in Example 1.

The procedure involves the in vivo treatment of Rs1h-defficient mice (Rs1h^{-/Y}). Control animals are also treated intrabulbar with non-specific peptide. For the purpose of prevention of retinoschisis, the animals under analgesic and anesthetic influence receive at day p12, p14, p21, p28, p35, p42, p49, p56 an intrabulbar injection of recombinant or not recombinant Rs1h or non-specific peptide. A control group of animals treated with buffer (intrabulbar injection of 0.003 ml) is also kept. Each group of experimental animals consists of 8 animals, the maximum injection volume/injection being 0.003 ml. On day p58 a bief-flash electroretinogram (ERG) and scanning-laser ophthalmoscopy are done, thereafter the animals are sacrificed by CO₂ inhalation.

The retinoschisis-specific pathology is examined by cone photoreceptor count, histology, electron microscopy.

### Example 3

### Intraretinal Application of Recombinant or not Recombinant Retinoschisin Protein Rs1h (A Polypeptide of 24 kD) in the Rs1h^{-/Y} Mouse Animal Model Described in Example 1.

The procedure involves the in vivo treatment of Rs1h-defficient mice (Rs1h^{-/Y}). Control animals are also treated intraretinal with non-specific peptide. For the purpose of prevention of retinoschisis, the animals under analgesic and anesthetic influence receive at day p12, p14, p21, p28, p35, p42, p49, p56 an intraretinal injection of recombinant or not recombinant Rs1h or non-specific peptide. A control group of animals treated with buffer (intraretinal injection of 0.003 ml) is also kept. Each group of experimental animals consists of 8 animals, the maximum injection volume/injection being 0.003 ml. On day p58 a bief-flash electroretinogram (ERG) and scanning-laser ophthalmoscopy are done, thereafter the animals are sacrificed by CO₂ inhalation.

The retinoschisis-specific pathology is examined by cone photoreceptor count, histology, electron microscopy.

### Example 4

### Systemic Application of Recombinant or not Recombinant Retinoschisin Protein Rs1h (A Polypeptide of 24 kD) in the Rs1h^{-/Y} Mouse: Animal Model Described in Example 1.

The procedure involves the in vivo treatment of Rs1h-defficient mice (Rs1h^{-/Y}). Control animals are also treated systemically with non-specific peptide. For the purpose of prevention of retinoschisis, the animals not under analgesic and anesthetic influence receive a daily (from p5-p56) i.v. injection into the tail vein of recombinant or not recombinant Rs1h or non-specific peptide. A control group of animals treated with buffer (i.v. injection of 0.03 ml) is also kept. Each group of experimental animals consists of 8 animals, the maximum injection volume/injection being 0.03 ml. On day p58 a bief-flash electroretinogram (ERG) and scanning-laser ophthalmoscopy are done, thereafter the animals are sacrificed by CO₂ inhalation.

The retinoschisis-specific pathology is examined by cone photoreceptor count, histology and electron microscopy.

### Example 5

### Intrabulbar application of retinoschisin protein Rs1h-specific or non-silencing dsRNAs in wt C57BL/6 mice.

The procedure involves the in vivo treatment of wt C57BL/6 mice. For the purpose of the induction of a retinoschisis-like phenotype or retinoschisis, the animals under analgesic and anesthetic influence receive at day p12, p14, p21, p28, p35, p42, p49, p56 an intrabulbar injection of Rs1h-specific or non-silencing dsRNAs (intrabulbar injection of 200 μg dsRNA/kg BW in 0.003 ml). A control group of animals treated with buffer (intrabulbar injection of 0.003 ml) is also kept. Each group of experimental animals consists of 8 animals, the maximum injection volume/injection being 0.003 ml. On day p58 a bief-flash electroretinogram (ERG) and scanning-laser ophthalmoscopy are done, thereafter the animals are sacrificed by CO₂ inhalation.

The retinoschisis-specific pathology is examined by cone photoreceptor count, histology, electron microscopy.

### EXAMPLE 6

### Intraretinal Application of Retinoschisin Protein Rs1H-specific or Non-silencing dsRNAs in wt C57BL/6 Mice.

The procedure involves the in vivo treatment of wt C57BL/6 mice. For the purpose of the induction of a retinoschisis-like phenotype or retinoschisis, the animals under analgesic and anesthetic influence receive at day p12, p14, p21, p28, p35, p42, p49, p56 an intraretinal injection of Rs1 h-specific or non-silencing dsRNAs (intraretinal injection of 200 μg dsRNA/kg BW in 0.003 ml). A control group of animals treated with buffer (intraretinal injection of 0.003 ml) is also kept. Each group of experimental animals consists of 8 animals, the maximum injection volume/injection being 0.003 ml. On day p58 a bief-flash electroretinogram (ERG) and scanning-laser ophthalmoscopy are done, thereafter the animals are sacrificed by CO₂ inhalation.

The retinoschisis-specific pathology is examined by cone photoreceptor count, histology, electron microscopy.

### EXAMPLE 7

### Systemic Application of Retinoschisin Protein Rs1H-Specific or Non-Silencing dsRNAs in wt C57BL/6 Mice

The procedure involves the in vivo treatment of wt C57BL/6 mice. For the purpose of the induction of a retinoschisis-like phenotype or retinoschisis, the animals not under analgesic and anesthetic influence receive a daily (from p5-p56) i.v. injection into the tail vein of Rs1h-specific or non-silencing dsRNAs (systemic injection of 200 μg dsRNA/kg BW in 0.03 ml). A control group of animals treated with buffer (i.v. injection of 0.03 ml) is also kept. Each group of experimental animals consists of 8 animals, the maximum injection volume/injection being 0.03 ml. On day p58 a bief-flash electroretinogram (ERG) and scanning-laser ophthalmoscopy are done, thereafter the animals are sacrificed by CO₂ inhalation.

The retinoschisis-specific pathology is examined by cone photoreceptor count, histology and electron microscopy.

### Methods

For further illustration, the methods mentioned in the examples above, concerning the generation and characterization of Rs1h-Deficient mice, supplement of protein Rs1h and the post transcriptional gene silencing, are described in the following sections.

Analgesia and Anesthesia of the Mice:

For systemic application, the animals are immobilized and the peptides are injected i.v. in the tail vein (maximal injection volume: 0.03 ml), where analgesia or anesthesia are refrained from, since this would put more stress on the animals than the i.v. injection itself. For intrabulbar and intraretinal injection (maximal injection volume: 0.003 ml) the animals are however subjected to short-term isoflurane inhalation anaesthesia and provided with Metamizole sodium for analgesic purposes. The animals are then kept in their accustomed animal cage surroundings. After completion of in vivo diagnosis with electroretinogram and scanning-laser ophthalmoscopy the animals are killed by CO₂ inhalation, enucleated and the eyes are studied histologically.

Generation of Rs1h-Deficient Mice:

CJ7 ES cells were electroporated and selected as described (Swiatek and Gridley (1993) Genes Dev. 7, 2071-2084). DNA was isolated from 300 colonies according to published methods (Ramirez-Solis et la. (1993) Methods Enzymol. 225, 855-878). Positive homologous recombination was identified by Southern blot analysis by using 5′ and 3′ probes external to the targeting construct generated with primer pairs rsm2F (5′-CAC ATT GGG ATT GTC ATC G-3′)/rsmint2R (5′-GGC TTC AGG AGT AGG GTA TC-3′) and rsm3′pF (5′-TGT AGC AAC CAT CCA ATA GG-3′)/rsm3′pR (5′-ATG TCC TCG TAT GTG CTA AG-3′), respectively, as well as by PCR with primer pairs rsm2F/lacZR2 (5′-CAA GGC OAT TAA GTT GGG TAA C-3′) and ssbpΛF2 (5′-AGA GCT CCG CGG CTC GAC TOT GCC TTC TAG TT-3′)/rsm3′pR (FIG. 1a and b). The injection of mutant ES cells into C57BL/6 blastocysts (Schrewe et la. (1994) Mech. Dev. 47, 43-51) resulted in five high-percentage coat-color chimeras, two of which exhibited germ-line transmission when bred to C57BL/6 females. Female F1 animals heterozygous for the Rs1h mutation were intercrossed with C57BL/6 mice to generate hemizygous male offspring.

Northern Blot Analysis:

Total RNA from murine eye cups was prepared by using standard techniques. Hybridization probes were generated by RT-PCR, with primers rsm4F/rsm6R encompassing exons 4 to 6 of the Rs1h gene and by excision of the recombinant lacZ gene. The fragments were randomly labeled in the presence of ³²PdCTP (3,000 Ci/mmol; 1 Ci=37 GBq).

Western Blot Analysis:

Polyclonal peptide antibody pAB-ap3RS1 (Molday et al. (2001) Invest. Ophthalmol. Visual Sci. 42, 816-825) was affinity purified from rabbit antiserum. Peroxidase-conjugated anti-rabbit IgG was used as a secondary antibody and visualized by using the enhanced chemiluminescence detection system (Amersham Pharmacia).

PCR Analysis of Temporal Expression of Rs1h

Analysis of temporal expression of Rs1h during retinal development in the mouse (from postnatal day P0 to P21) was done by RT-PCR analysis of Rs1h with primers rsm2F (5′-CAC ATT GGG ATT GTC ATC G-3′) and rsm6R (5′-GAT GAA GCG GGA AAT GAT GG-3′). The retina-specific cone-rod homeo box-containing gene (CRX) and β-actin were used as control reactions to test for cDNA integrity at all stages of development tested. Primer sequences for CRX were Crx-F: 5′-GTCCCCCACCTCCTTGTCAG-3′ and Crx-R: 5′-CCT CAA GTT CCC AGC AAT CC-3′, for β-actin XAHR20: 5′-ACC CAC ACT GTG CCC ATC TA-3′ and XAHR17: 5′-CGG AAC CGC TCA TTG CC-3′. PCR conditions were TA 58° C., 1,5 mM MgCl₂, 4% Formamide, Rs1: rsm2F/rsm6R, 28 cycles, CRX: Crx-F/Crx-R, 19 cycles, β-actin: XAHR20/XAHR17, 19 cycles.

Electroretinogram:

Electroretinograms (ERGs) were obtained according to reported procedures (Seeliger et al. (2001). Nat. Genet. 29, 70-74). Briefly, before anesthesia with ketamine (66.7 mg/kg), xylazine (11.7 mg/kg), and atropine (1 mg/kg), the pupils of dark-adapted mice were dilated.

Alternatively, before short-term isoflurane isolation anaesthesia, the pupils of dark-adapted mice were dilated. The ERG equipment consists of a Ganzfeld bowl, a DC amplifier, a PC-based control and a recording unit (Toennies Multiliner Vision, Hoechberg, Germany). Band-pass filter cut-off frequencies are 0,1 and 3,000 Hz. Single flash recordings are obtained both under dark-adapted (scotopic) and light-adapted (photopic) conditions. Light adaption before the photopic session is performed with a background illumination of 30 cd/m² for 10 min. Single fash stimulus intensities were increased from 10⁻⁴ cd·s/m² to 25 cd·s/m² and divided into 10 steps of 0,5 and 1 log cd·s/m². Ten responses were averaged with an inert-stimulus interval of either 5 s or 17 s (for 1, 3, 10, 25 cd·s/m²).

Scanning-laser Ophthalmoscopy:

Fundus imaging is performed with an HRA scanning-laser ophthalmoscope (SLO) with an infrared wavelength of 835 nm (Heidelberg Instruments, Heidelberg, Germany). The confocal diaphragm of the SLO allows imaging of different planes of the posterior pole, ranging from the surface of the retina down to the retinal pigment epithelium (RPE) and the choroid. Different planes can be viewed sequentially by varying the focus by about ±20 diopteres.

Cone Photoreceptor Count:

The relative number of cone photoreceptor cells is estimated from counts of total cone opsin-labeled cells (JH 492 and JH455) in a series of retinal section through the eyes of control and treated mice. Usually, a series of retinal sections through the eyes of three wt and Rs1h /Y mice, respectively, are being used.

Histology and Electron Microscopy:

Two-month-old control and treated mice are perfusion-fixed via the heart with Ito's fixative (Ito and Karnovsky (1968) J. Cell Biol. 39, 168A-169A). After enucleation, the eyes are biseceted equatorially and immersed in the same fixative for 24 h. after fixation, the samples are washed overnight in cacodylate buffer, post-fixed with OsO4, dehydrated, and embedded in epon (Roth, Karlsruhe, Germany). Semithin sections (1 μm) are stainded with toluidin blue for serial histological analysis. Ultrathin sections are stained with uranyl acetate and lead citrate and viewed with an EM 902 electron microscope (Zeiss, Mainz, Germany) After removal, the eyes are fixed in 4% formalin/PBS solution for 24 hours. Using standard methods, the fixed samples are subsequently dehydrated in a series of increasing alcohol and embedded in paraffin. With the aid of a microtome, standard 5 to 12 μm serial slices are produced, stretched in a heated water bath and transferred to a polylysin-coated cover slip. The sections are then dried in an incubator for 2 hours at a temperature of 52° C. The dried sections are deparaffinated in xylol, transferred to a decreasing series of alcohol followed by Tris/HCl pH 7.4. After blocking, the sections are incubated for 2 hours with primary anti-eGFP antiserum (polyclonal goat anti-eGFP antiserum, Santa Cruz No. sc-5384). Detection occurs by means of immunofluorescence staining by using a Cy2-conjugated rabbit anti-goat IgG (Dianova, No. 305-225-045). The samples are embedded and then mounted for microscopy with an Eclipse TE-2000-S microscope (Nikon), equipped with a 20× and 40×/1.3 objective. The spontaneous, eGFP-specific fluorescence in deparaffinated, untreated sections is analyzed using a fluorescence microscope.

Immunofluorescence Labeling:

For immunofluorescence studies, retina dissected from two-month-old mutant and wt mice were paraformaldehyde-fixed for 1-2 h and subsequently rinsed in PBS containing 10% (wt/vol) sucrose. Cryosections were blocked with PBS containing 0.2% Triton X-100 (PBS-T) and 10% (vol/vol) goat serum for 20 min and labeled overnight with the primary antibody. The samples then were rinsed in PBS and labeled for 1 h with the secondary antibody conjugated to Cy3 (red) or Alexi 488 (green) (Jackson ImmunoResearch). The Rs1 3R10 monoclonal antibody was produced from a mouse immunized with a glutathione S-transferase fusion protein containing the LSSTEDEGEDPWYQKAC peptide, corresponding to amino acids 22-39 of the human RS1 precursor protein (Sauer et al. (1997) Nat. Genet. 17, 164-170). Cell-specific antibodies used were Rho 1D4 monoclonal antibody to rhodopsin (MacKenzie and Molday (1982) J. Biol. Chem. 157, 7100-7105), Mab 115A10 monoclonal antibody to rat olfactory bulb (a generous gift of Shinobu C. Fujita, Mitsubishi Kasei Institute of Life Sciences, Tokyo; Onoda and Fujita (1987) Brain Res. 416, 359-363). JH 492 polyclonal antibody to red/green (middle wavelength) cone opsin and JH 455 blue (short wavelength) cone opsin (a generous gift of J. Nathans, Johns Hopkins University, Baltimore), PAN-SAP polyclonal antibody, (a generous gift of Craig C. Garner, Department of Neurobiology, Univ. of Alabama, Birmingham) and 7E3-1B8 monoclonal antibody (Affinity BioReagents, Golden, Colo.) to the postsynaptic density protein 95 (PSD95), and CRALBP polyclonal antibody to cellular retinal binding protein (a generous gift of Jack Saari, Department of Ophthalmology, Univ. of Washington, Seattle; ref: Bunt-Milam and Saari (1983) J. Cell Biol. 97, 703-712). The PAN-SAP and 7E3-1B8 antibodies showed the same labeling pattern, although the PAN-SAP stained mouse retina more intensely.

Retinoschisin Rs1h-specific Polypeptide Sequence:

MPHKIEGFFLLLLFGYEATLGLSSTEDEGEDPWYQKACKCDCQVGANALWSAGATSLDCIPE
CPYHKPLGFESGEVTPDQITCSNPEQYVGWYSSWTANKARLNSQGFGCAWLSKYQDSSQWLQ
IDLKEIKVISGILTQGRCDIDEWVTKYSVQYRTDERLNWIYYKDQTGNNRVFYGNSDRSSTV
QNLLRPPIISRFIRLIPLGWHVRIAIRMELLECASKCA

dsRNA Constructs:

For the design of the dsRNA molecules, sequences of the type AA(N19)TT (where N represents any nucleotide) were selected from the sequence of the target mRNA, in order to obtain 21 nucleotide (nt) long sense and antisense strands with symmetrical 3′-overhangs of two nucleotides in length. In the 3′-overhangs, 2′-deoxy-thymidine was used instead of uridine. In order to ensure that the dsRNA molecules are exclusively directed against the target gene, the chosen dsRNA sequences are tested against the mouse genome in a BLAST analysis. The 21-nt RNA molecules are synthesized chemically and purified. For the duplex formation, 100 μg of the sense and antisense oligoribonucleotides each are mixed in 10 mM Tris/HCl, 20 mM NaCl (pH 7.0) and heated to 95° C. and cooled to room temperature over a period of 18 hours. The dsRNA molecules are precipitated from ethanol and resuspended in sterile buffer (100 mM potassium acetate, 30 mM HEPES-KOH, 2 mM magnesium acetate, pH 7.4). The integrity and double strand character of the dsRNA are verified by gelelectrophoresis. Alternatively, the dsRNA molecules are obtained from commercial suppliers. The sequences of the target genes and the corresponding dsRNA molecules are as follows:

| | |
|---|---|
| Retinoschisin Rs1h-specific dsRNA | |
| DNA target sequence: | 5′ AAGTATCAGGACAGCAGCCAG |
| | (Acc. No. NM_011302) |
| dsRNA (sense) | 5′ r(GUAUCAGGACAGCAGCCAG)d(TT) |
| dsRNA (antisense) | 5′ r(CUGGCUGCUGUCCUGAUAC)dTT |
| non-silencing dsRNA, control | |
| DNA target sequence: | 5′ AATTCTCCGAACGTGTCACGT |
| dsRNA (sense) | 5′ r(UUCUCCGAACGUGUCACGU)d(TT) |
| dsRNA (antisense) | 5′ r(ACGUGACACGUUCGGAGAA)d(TT) |

### Gene Therapy of X-linked Juvenile Retinoschisis

As another examples a recombinant adeno-associated virus (rAAV)-based gene therapy approach is performed in the mouse model for X-linked juvenile retinoschisis with the aim to determine the efficacy of RS1 somatic gene transfer to the retinoschisin-deficient mouse retina. The experiments are performed in two phases. Phase I aims at examining fundamental parameters such as toxicity, immune response or rate of transduction of various virus preparations (promoter constructs) after defined time points post-injection. In a second phase, the spatial and temporal effects of transgene activity in the diseased retina of the Rs1h knock-out mouse are determined. Together, the results of phase I and phase II experiments allow to assess the feasibility and efficacy of AAV-mediated gene therapy for the X-linked condition of juvenile retinoschisis.

### Example 8

### Plasmid Construction, Recombinant AAV-RS1 Virus Preparation and Surgical Delivery

For routine AAV vector production, a two plasmid co-transfection procedure and cell factories of adherent. H293 cells is employed. Calcium phosphate is used to introduce the AAV vector plasmid together with a helper plasmid, pDG, at a 1:1 molar ratio. pDG is a combined non-packaging AAV-Ad helper plasmid (Grimm et al. 1998). The plasmid encodes four adenoviral open reading frames, not including the E1-region, which are necessary to complement AAV vector production. As such, packaging of rAAV is restricted to E1-complementing cell lines such as H293. Additionally, pDG encodes the AAV rep and cap genes, also necessary for rAAV production. The rep gene is under the control of a weak MMTV LTR promoter thereby reducing Rep78/68 protein levels. This produces a 5-10 fold increase in viral yield and reduces the chance of a productive illegitimate recombination event that might generate wild type AAV contamination. The UF series of AAV vectors that contain only the 145 bp TR sequences and convenient restriction sites is also used for testing the ability of different promoters to drive the expression of the desired cDNA flanked by NotI sites (Zolotukhin et al. 1996). All AAV vectors are made in this pTR-UF background. More than 140 separate cell factory preparations of AAV serotype 1, 2 or 5 vectors are made, most purified by the iodixanol-FPLC method (see below).

The protocol for the purification and concentration of small lots of AAV virus is based on partial purification of the initial freeze/thaw host cell lysate by iodixanol gradient fractionation, followed by Q-column FPLC (Hauswirth et al. 2000; Zolotukhin et al. 1999). AAV vectors purified by this method appear to be at least 99.9% pure (Hauswirth et al. 2000). This method is currently used for all individual cell factory preparations (about 10⁹ host cells). The final vector stock is titered by infectious center, slot blot and/or Taqman assays. 100-200 infectious units (iu) per cell are routinely obtained. For a cell factory preparation, this means that the final yield of AAV is approximately 10¹¹ iu or approximately 5×10¹² DNase resistant particles per ml. The iodixanol-Qcolumn purification yields low particle to infectivity (P/I) ratios, typically 20-50 and rarely above 100.

The recombinant AAV vector to be evaluated in this proposal contains a 472-bp mouse opsin promoter (Mops) mapping between −386 to +86, that supports strong, rod photoreceptor-specific expression in the mouse (Flannery et al. 1997). It has been placed immediately upstream to the human RS1 cDNA. Because AAV serotypes can exhibit somewhat different transduction efficiencies in the retina (Auricchio et al. 2001; Rabinowitz et al. 2002), AAV serotypes 1, 2 and 5 containing the Mops-RS1 insert are made and tested.

For the purpose of determining in vivo expression levels and phenotypic rescue in the knock-out mouse, each vector containing the RS1 cDNA is injected into the subretinal space of normal or Rsh1 knock-out mice under general and local (corneal) anesthesia. At injection, ages of the mice will range from P1 to two months, the animals will have an aperture made through the inferior cornea with a beveled 28-gauge needle. Subretinal injection of 1 μl is then made by inserting a blunt 32-gauge needle through the opening and delivering the vector suspension into the subretinal space of the inferior hemisphere. Injections are performed under direct observation with an operating microscope, and the subretinal location of the injection visualized. This anterior approach for subretinal injection results in the occasional induction of cataracts due to contact with the needle. Animals with cataracts are not used for analysis. Approximately 10¹⁰ particles (2×10⁸ infectious units) are delivered in a volume of 1 μl to the right eye. The contralateral eye is injected with the same volume of the relevent reference vector, either AAV-Mops-GFP of the matching serotype (1, 2 or 5) or carrier PBS. Data from such test-control pairs of eyes allows groups of 10 animals to supply sufficient data for statistical significance (La Vail et al. 2000; Lewin et al. 1998; Mori et al. 0.2002; Raisler et al. 2002). AAV2-CBA-GFP treated eyes is an important control to rule out any non-cDNA “vector” effect.

### Example 9

### Transgene Expression and Long-term Persistence

The location, magnitude and persistance of AAV-mediated RS1 expression in the murine eye is monitored by RT-PCR as well as immunolabeling studies. For RT-PCR, the eyecups are dissected into the four quadrants and retina and RPE/choroid tissue are removed for total RNA isolations. PCR amplification are done in first strand cDNA with forward and reverse primers encompassing the full length transcript of RS1. For immunolabeling studies, retinal cryosections are stained with polyclonal antibody pAB-ap3RS1 (Molday et al. 2001) or monoclonal antibody Rs1 3R10S; see Example 1. Animals are sacrificed after 14, 28, 56, 120, 240, and 550 days post-injection. Although only rod-specific vector transduction is expected, to identify the specific cell type expressing retinoschisin, double labeling studies with a set of retinal cell-specific antibodies are performed if necessary.

### Example 10

### Methods of Scanning-laser Ophthalmoscopy (SLO) and Electroretinography (ERG).

In vivo fundus imaging is performed with a scanning laser ophthalmoscope using a scanning frequency of 50 Hz and an infrared wavelength of 780 nm. The confocal diaphragm of the SLO allows imaging of different planes of the posterior pole, ranging from the surface of the retina down to the retinal pigment epithelium (RPE) and the choroid. As was shown in Example 1, SLO is well suited to macromorphologically monitor the cyst-like pathology in the living animal.

Similarly, the scotopic and photopic responses of the ERG recordings are a sensitive and non-invasive measure of inner retinal function. Single flash recordings are obtained both under dark-adapted (scotopic) and light-adapted (photopic) conditions. The scotopic ERG in the Rs1h knock-out mouse shows a dramatic loss of the positive b-wave, which is mostly shaped by the neurons of the inner retina. In contrast, the negative a-wave representing both inner and outer retinal components, is relatively preserved; see Example 1. Under photopic conditions, ERG responses are virtually absent, suggesting a profound dysfunction of the cone system. The SLO and ERG recordings are performed in 4-6 animals, one eye treated, one eye control analyzed over the same time course as above.

### Example 11

### Histology, Electron Microscopy and Immunofluorescence Labeling

Histologic examination of retinal sections from Rs1h knock-out mice show a pronounced disorganization of the INL and ONL. This is accompanied by a significant reduction in the number of photoreceptor nuclei generally noticeable at approximately 4-6 weeks postnatal; see Example 1. The thickness of the ONL is determined as a measure of photoreceptor nuclei after DAPI staining of retinal sections. In addition, cone photoreceptor count is done semi-quantitatively by estimating the total cone opsin-labeled cells (using opsin antibodies JH492 and JH455).

By electron microscopy increased extracellular spaces in the region of photoreceptor ribbon synapses and larger extracellular gaps are present between individual photoreceptor terminals and the perikarya of the INL; see Example 1. Therefore, effects of AAV-RS1 injections are monitored at the ultrastructural level.

To further determine the specific retinal cell types affected by AAV-RS1 transgene expression, cryosections from wildtype and injected Rs1h knock-out retinae are labeled with cell-specific antibodies for analysis by immunofluorescence microscopy. A large selection of monoclonal and polyclonal antibodies labeling various retinal- and RPE-relevant proteins in the mouse eye can be used such as Abca4, Peripherin-2, Rom-1, Timp3, rhodopsin, collagen II, collagen IV, collagen IV, collagen XVIII, G protein-coupled receptor-75, Vmd2, Mpp4, Neto-1, Wdr17, Psd-95, Cask, Rs1, Glt1, recoverin, or cone opsin.

### Discussion

For several human diseases animal models have been tried to be established. While disease related genes and techniques for producing transgenic animals are generally available, it nevertheless is not predictable whether for example introduction or knock-out of one gene establishes a phenotype in the transgenic animal that comes close enough to that clinically observed in humans and/or whether the animal is suitable for predicting therapy systems for treating the human disease.

The present invention discloses the generation and characterization of an Rs1h^{-/Y} knock-out mouse and could surprisingly demonstrate that this mouse line is a valuable model for RS with a retinal phenotype closely paralleling that of the human condition. Although the murine retina lacks a macular organization, those findings in the Rs1h^{-/Y} mutant animals demonstrate that mice can still be useful for modeling human diseases that display a primary macular phenotype such as RS.

The major pathology in the retina of the retinoschisin-deficient mouse seems to be a generalized disruption of cell layer architecture, most evident in the loss of integrity of the OPL/INL and an irregular displacement of cells in various retinal layers. Functionally, ERG recordings point to severe impairment of bipolar cell-associated pathways and a loss of photoreceptors that is more pronounced in cone than in rod pathways. This finding also is supported by immunofluorescence labeling studies. Rod staining indicates a generalized decrease in cell number, whereas cone labeling demonstrates a more striking cell loss as well as a defect in the targeting of cone opsin to the outer segments. Similarly, in the organization of the bipolar cell layer, there are clear abnormalities, which may be instrumental to the relative b-wave attenuation demonstrated by ERG recordings in human (Green and Kapousta-Bruneau (1999) Visual Neurosci. 16, 727-741; Lei and Perlman (1999) Visual Neurosci. 16, 743-754) and the Rs1h^{-/Y} mouse.

The observed distortion of retinal layers in the Rs1h^{-/Y} mouse could be explained by the loss of cell-cell and/or cell-matrix interactions, both of which are thought to be mediated by the discoidin domain of retinoschisin (Baumgartner et al. (1998) Protein Sci. 7, 1626-1631; Vogel (1999) FASEB J. 13, S77-SR2). The functional importance of this domain also is reflected by the mutational profile determined in more than 320 RS patients worldwide; see the internet page of dmdl.nl/rs/rshome.htm on the world wide web. Of the 125 distinct sequence changes identified so far, 101 (81%) occur in exons 4 to 6 that encode the discoidin motif and likely impair defined functional aspects of this domain. Two types of discoidin-mediated binding can be envisioned. A collagen-discoidin interaction (Shrivastava et al. (1997) Mol. Cell 1, 25-34; Vogel et al. (1997) Mol. Cell 1, 13-23) could anchor cells into an extracellular matrix scaffold or mediate transmembrane-signaling processes. For example, in a direct ligand-binding assay, the introduction of amino acid mutations in the discoidin domain of the discoidin domain receptor 1 (DDR1) at positions homologous to several retinoschisin mutations affects collagen binding and/or receptor phosphorylation of DDR1 (Curat et al. (2001) J. Biol. Chem. 276, 45952-45958). These experiments suggest that binding of discoidin domains to (collagenous) components of the extracellular matrix could be a more general property of these modules. Binding to membrane-anchored carbohydrate residues could represent an alternative mode of discoidin-mediated function. Such interactions would facilitate cell-td-cell contacts and are thought to play a critical role in the activation of the blood clotting cascade on platelet membrane surfaces (Kim et al. (2000) Biochemistry 39, 1951-1958; Kiight et al. (1999) Cardiovasc. Res. 41, 450-457).

One of the most important cellular contacts in the retina occurs at the synapse, where retinoschisin is ordinarily present in high amounts. The present finding of loss of the synaptic MAGUK protein PSD-95 in the IPL of the Rs1h deficient mouse and defects in its translocation to the OPL points to a direct or indirect role of retinoschisin in the proper assembly and stabilization of this region of the cell. This finding also is supported by transmission electron microscopy revealing atypical ribbon synapse formation at the photoreceptor terminals of Rs1h^{-/Y} mice. Failure to establish or maintain the proper synaptic connections could lead to subsequent photoreceptor cell death, a phenomenon that also has been reported in an animal model transgenic for P347L rhodopsin (Blackmon et al. (2000) Brain Res. 885, 53-61).

The Rs1h^{-/Y} mouse shares several diagnostic features with human RS, including the typical “negative ERG” response and the development of cystic structures within the inner retina, followed by a dramatic loss of photoreceptor cells. Therefore, it is concluded that the Rs1h^{-/Y} mouse represents an important model system for further investigations into the molecular mechanisms underlying the cellular disorganization of the retinal structure. This model is particularly useful for the evaluation of the role of retinoschisin in the assembly and stabilization of synaptic contacts.

Furthermore, while it was tempting to speculate that of Rs1h^{-/Y} mice might be useful as experimental system for therapeutic approaches in X-linked juvenile retinoschisis, the present invention could surprisingly show that those mice can be used as tool for establishing therapies for the treatment of retinoschisis, in particular therapeutic protein and gene transfer as well as regimens for safe and appropriate medical intervention in the prevention and treatment of retionschisis.

### REFERENCES

Auricchio A, Kobinger G, Anand V, Hildinger M, O'Connor E, Maguire A M, Wilson J M, Bennett J. Exchange of surface proteins impacts on viral vector cellular specificity and transduction characteristics: the retina as a model. Hum Mol Genet 10: 3075-3081 (2001)

Baumgartner S, Hofmann K, Chiquet-Ehrismann R, Bucher P. The discoidin domain family revisited: new members from prokaryotes and a homology-based fold prediction Protein Sci 7: 1626-1631 (1998)

Condon G P, Brownstein S, Wang N S, Kearns J A, Ewing C C. Congenital hereditary (juvenile X-linked) retinoschisis. Histopathologic and ultrastructural findings in three eyes. Arch Ophthalmol 104: 576-583 (1986)

De La Chapelle A, Alitalo T, Forsius H. X-linked juvenile retinoschisis, In Molecular Genetics of inherited eye diseases (eds Wright A F and Jay B). Harwood Academic Publishers Switzerland, 339-357 (1994)

Flannery J G, Zolotukhin S, Vaquero M I, LaVail M M, Muzyczka N, Hauswirth W W. Efficient photoreceptor-targeted gene expression in vivo by recombinant adeno-associated virus. Proc Natl Acad Sci USA 94: 6916-6921 (1997)

Gehrig A E, Warneke-Wittstock R, Sauer C G, Weber B H F. Isolation and characterization of the murine X-linked juvenile retinoschisis (Rs1h) gene. Mamm Genome 10: 303-307 (1999)

George N D, Yates J R, Moore A T. Clinical features in affected males with X-linked retinoschisis. Arch Ophthalmol 114: 274-280 (1996)

Grayson C, Reid S N, Ellis J A, Rutherford A, Sowden J C, Yates J R, Farber D B, Trump D. Retinoschisin, the X-linked retinoschisis protein, is a secreted photoreceptor protein, and is expressed and released by Weri-Rb1 cells. Hum Mol Genet 9: 1873-1879(2000)

Grimm D, Kern A, Rittner K, Kleinschmidt J A. Novel tools for production and purification of recombinant adenoassociated virus vectors. Hum Gene Ther 9: 2745-2760 (1998)

Hauswirth W W, Lewin A S, Zolotukhin S, Muzyczka N. Production and purification of recombinant adeno-associated virus. Methods Enzymol 316: 743-761 (2000)

LaVail M M, Yasumura D, Matthes M T, Drenser K A, Flannery J G, Lewin A S, Hauswirth W W. Ribozyme rescue of photoreceptor cells in P23H transgenic rats: long-term survival and late-stage therapy. Proc Natl Acad Sci USA 97: 11488-11493 (2000)

Lewin A S, Drenser K A, Hauswirth W W, Nishikawa S, Yasumura D, Flannery J G, LaVail M M. Ribozyme rescue of photoreceptor cells in a transgenic rat model of autosomal dominant retinitis pigmentosa. Nat Med 4:967-971 (1998)

Molday L L, Hicks D, Sauer C G, Weber B H, Molday R S. Expression of X-linked retinoschisis protein RS1 in photoreceptor and bipolar cells. Invest Ophthalmol Vis Sci 42: 816-825 (2001)

Mori K, Gehlbach P, Yamamoto g, Duh E, Zack D J, Li Q, Berns K I, Raisler B J, Hauswirth W W, Campochiaro P A. AAV-mediated gene transfer of pigment epithelium-derived factor inhibits choroidal neovascularization. Invest Ophthalmol Vis Sci 43: 1994-2000 (2002)

Rabinowitz J E, Rolling F, Li C, Conrath H, Xiao W, Xiao X, Samulski R J. Cross-packaging of a single adeno-associated virus (AAV) type 2 vector genome into multiple AAV serotypes enables transduction with broad specificity. J Virol 76: 791-801 (2002)

Raisler B J, Berns K I, Grant M B, Beliaev D, Hauswirth W W. Adeno-associated virus type-2 expression of pigmented epithelium-derived factor or Kringles 1-3 of angiostatin reduce retinal neovascularization. Proc Natl Acad Sci USA 99: 8909-8914 (2002)

Robson, J. G. & Frishman, L. J. (1998) Doc. Ophthalmol. 95, 187-215

Roesch M T, Ewing C C, Gibson A E, Weber B H F. The natural history of X-linked retinoschisis. Can J Ophthalmol 33: 149-158.(1998)

Sauer C G, Gehrig A, Warneke-Wittstock R, Marquardt A, Ewing C C, Gibson A, Lorenz B, Jurklies B, Weber B H F. Positional cloning of the gene associated with X-linked juvenile retinoschisis. Nature Genet 17: 164-170 (1997)

Weber B H F, Schrewe H, Molday L L, Gehrig A, White K L, Seeliger M W, Jaissle G B, Friedburg C, Tamm F., Molday R S. Inactivation of the murine X-linked juvenile retinoschisis gene, Rs1h, suggests a role of retinoschisin in retinal cell layer organization and synaptic structure. Proc Nat Acad Sci USA 99: 6222-6227 (2002)

Zolotukhin S, Byrne B, Mason E, Zolotukhin I, Potter M, Chesnut K, Summerford C, Samulski R, Muzyczka N. Recombinant adeno-associated virus purification using novel methods improves infectious titer and yield. Gene Ther 6: 973-985 (1999)

Zolotukhin S, Potter M, Hauswirth W W, Guy J, Muzyczka N. A “humanized” green fluorescent protein cDNA adapted for high-level expression in mammalian cells. J Virol 70: 4646-4654 (1996)

1. A transgenic non-human animal comprising a recombinant nucleic acid molecule the presence of which leads to inactivation of the expression of a gene orthologous to the human RS1 gene, and wherein said animal displays one or more clinical symptoms of X-linked juvenile retinoschisis (RS).

2. The transgenic animal of claim 1, wherein the animals ortholog RS1 gene has been inactivated.

3. The transgenic animal of claim 2, wherein the endogenous RS1 gene is disrupted by a polynucleotide encoding a fragment of the RS1 gene in combination with a selection marker.

4. The transgenic animal of claim 3, wherein said polynucleotide encoding a reporter gene and a selectable marker gene is flanked by genomic regions of the RS1 gene within the same open reading frame.

5. The transgenic animal of claim 3, wherein the reporter gene is the LacZ gene.

6. The transgenic animal of claim 3, wherein the selectable marker gene confers an antibiotic resistance, preferably a neomycine resistance.

7. The transgenic animal of claim 4, wherein the flanking regions of the RS1 gene are exon 3 upstream and intron 3 plus exon 4 downstream of the reporter and marker gene, respectively.

8. The transgenic animal of claim 1, wherein the animal is a mammal.

9. The transgenic animal of claim 8, wherein the animal is a rodent.

10. The transgenic animal of claim 9, wherein the animal is a mouse.

11. The transgenic animal of claim 1, wherein the animal displays symptoms of macular degeneration.

12. The transgenic animal of any one of claim 1, wherein the animal develops small cyst-like structures in the inner retina.

13. The transgenic animal of claim 1, wherein the dark-adapted ERG measurements show a dramatic loss of the positive b-wave when compared to control animals and the light adapted ERG responses are virtually absent.

14. The transgenic animal of claim 1, wherein the rod function is not impaired.

15. The transgenic animal of claim 1, wherein the retinal layers are disorganized.

16. A method of producing a transgenic non-human animal displaying one or more clinical symptoms of X-linked juvenile retinoschisis (RS) comprising:
(a) introducing a nucleic acid construct comprising at least part of the RS1 gene interrupted in frame by a nucleic acid sequence encoding a reporter gene and a selectable marker gene into an embryo of a non-human animal;
(b) implanting the embryo into a female foster animal of the same species and allow it to develop normally until birth;
(c) screening the offsprings for presence of the nucleic acid construct in the germline; and optionally
(d) mating those offsprings whose germline contains the nucleic acid construct.

17. The method of claim 16, wherein said nucleic acid construct is introduced into an ES cell, screened for the correct integration locus within the RS1 gene and is then transferred into said embryo preferably by microinjection.

18. The method of claim 16 wherein said nucleic acid is introduced into a fertilized egg of said animal, preferably by microinjection and allowing the egg to divide into an early embryo, which is the transferred into said foster animal.

19. A method of producing a non-human animal displaying one or more clinical symptoms of X-linked juvenile retinoschisis (RS) comprising introducing one or more nucleic acid molecules comprising a nucleotide sequence derived from the human RS1 gene or from a corresponding ortholog or a vector encoding and capable of expressing such nucleic acid molecules into a cell or tissue of the animal, wherein said nucleic acid molecule is capable of provoking the degradation of the corresponding mRNA encoding RS1 or an orthologous gene product.

20. The method of claim 19, wherein said nucleic aicd molecule comprises a double-stranded oligoribonucleotide (dsRNA).

21. An animal obtainable by the method claim 19.

22. The animal of claim 21 which is mouse.

23. A polynucleotide as defined in Claim 3.

24. A nucleic acid molecule as defined in claim 19

25. A method of screening test therapies as potential prevention or treatment of retinoschisis comprising determining the time frame for the onset or development of one or more of the clinical symptoms displayed by the animal of claim 1

26. The method of claim 25, wherein said symptoms are determined by Scanning-Laser Ophthalmoscopy, Electroretinogram, Histology and Electron Microscopy, Immunofluorescence Labeling, and/or Cone Photoreceptor Count.

27. The method of claim 25 further comprising:
(a) administering a composition comprising a test compound known to be capable to compensate for the loss of expression of the RS1 gene or for the activity of the RS1 gene product to the animal at different times and/or dosages within one of the identified time frames;
(b) monitoring said animal for alleviating the symptoms; and
(c) determine the optimal administration time, release and/or dosage regimen.

28. The method of claim 27, wherein said test compound is formulated in a composition for retarded release and/or release at predetermined time after administration of the composition.

29. The method of claim 27, wherein the therapy is a gene therapy.

30. The method of claim 27, wherein the therapy is a protein replacement therapy.

31. A method of screening and/or isolating compounds having therapeutic activity in the treatment of retinal disorders comprising:
(a) administering a test compound to a transgenic non-human animal of claim 1; and
(b) monitoring said animal to determine if the compound is alleviating the symptoms.

32. The method of claim 31, wherein said test compound is administered in a time, and/or dosage regimen and/or retarded release formulation determined according to a method comprising:
(a) administering a composition comprising a test compound known to be capable to compensate for the loss of expression of the RS1 gene or for the activity of the RS1 gene product to the animal at different times and/or dosages within one of the identified time frames;
(b) monitoring said animal for alleviating the symptoms; and
(c) determine the optimal administration time, release and/or dosage regimen.

33. A pharmaceutical composition comprising a compound identified or isolated according to the method of claim 31, wherein said composition is formulated so to release the compound at the time and/or in dosage determined according to a method comprising:
(a) administering a composition comprising a test compound known to be capable to compensate for the loss of expression of the RS1 gene or for the activity of the RS1 gene product to the animal at different times and/or dosages within one of the identified time frames;
(b) monitoring said animal for alleviating the symptoms; and
(c) determine the optimal administration time, release and/or dosage regimen.

34. A method of prevention or treatment of retinoschisis comprising administering to a subject in need thereof a therapeutically effective amount of a compound capable to compensate for the loss of expression of the RS1 gene or for the activity of the RS1 gene product to the subject.

35. The method of prevention or treatment of retinoschisis comprising administering to a subject in need thereof a therapeutically effective amount of a compound capable to compensate for the loss of expression of the RS1 gene or for the activity of the RS1 gene product to the subject, wherein said compound is administered in a time and/or dosage regimen determined according a method of claim 25.

36. The method of claim 35, wherein said compound is wild type RS1 protein.

37. The method of claim 35, wherein said compound is a recombinant RS1 protein or functional derivative or analogue thereof.

38. The method of claim 34, wherein said compound is a recombinant nucleic acid molecule encoding RS1 protein or a functional derivative or analogue thereof.

39. The method of claim 38, wherein said recombinant nucleic acid molecule is gene transfer vector.

40. The method of claim 39, wherein said recombinant nucleic acid molecule is a recombinant adeno-associated virus (rAAV) based gene therapy vector.

41. The method of claim 38, wherein the expression of said RS1 protein or functional derivative or analogue thereof is under the control of the opsin promoter.

42. A method for the prevention or treatment of retinoschisis comprising surgical intervention in a time frame determined according to the method of claim 25.


SEQUENCE LISTING (18 sequences)

SEQ ID NO: 1 - DNA, 19 bp, Artificial
  source (1)..(19): Description of artificial sequence Primer
cacattgggattgtcatcg

SEQ ID NO: 2 - DNA, 20 bp, Artificial
  source (1)..(20): Description of artificial sequence Primer
ggcttcaggagtagggtatc

SEQ ID NO: 3 - DNA, 20 bp, Artificial
  source (1)..(20): Description of artificial sequence Primer
tgtagcaaccatccaatagg

SEQ ID NO: 4 - DNA, 20 bp, Artificial
  source (1)..(20): Description of artificial sequence Primer
atgtcctcgtatgtgctaag

SEQ ID NO: 5 - DNA, 22 bp, Artificial
  source (1)..(22): Description of artificial sequence Primer
caaggcgattaagttgggtaac

SEQ ID NO: 6 - DNA, 32 bp, Artificial
  source (1)..(32): Description of artificial sequence Primer
agagctccgcggctcgactgtgccttctagtt

SEQ ID NO: 7 - DNA, 20 bp, Artificial
  source (1)..(20): Description of artificial sequence Primer
gatgaagcgggaaatgatgg

SEQ ID NO: 8 - DNA, 20 bp, Artificial
  source (1)..(20): Description of artificial sequence Primer
gtcccccacctccttgtcag

SEQ ID NO: 9 - DNA, 20 bp, Artificial
  source (1)..(20): Description of artificial sequence Primer
cctcaagttcccagcaatcc

SEQ ID NO: 10 - DNA, 20 bp, Artificial
  source (1)..(20): Description of artificial sequence Primer
acccacactgtgcccatcta

SEQ ID NO: 11 - DNA, 17 bp, Artificial
  source (1)..(17): Description of artificial sequence Primer
cggaaccgctcattgcc

SEQ ID NO: 12 - protein, 224 aa, Mus musculus
  source (1)..(224): Retinoshisin Rsh1-specific polypetide
MPHKIEGFFLLLLFGYEATLGLSSTEDEGEDPWYQKACKCDCQVGANALWSAGATSLDCIPECPYHKPLGFESGEVTPDQITCSNPEQYVGWYSSWTANKARLNSQGFGCAWLSKYQDSSQWLQIDLKEIKVISGILTQGRCDIDEWVTKYSVQYRTDERLNWIYYKDQTGNNRVFYGNSDRSSTVQNLLRPPIISRFIRLIPLGWHVRIAIRMELLECASKCA

SEQ ID NO: 13 - DNA, 21 bp, Artificial
  source (1)..(21): Description of artificial sequence DNA target sequence
aagtatcaggacagcagccag

SEQ ID NO: 14 - DNA, 21 bp, Artificial
  source (1)..(21): Description of artificial sequence dsRNA
guaucaggacagcagccagtt

SEQ ID NO: 15 - DNA, 21 bp, Artificial
  source (1)..(21): Description of artificial sequence dsRNA
cuggcugcuguccugauactt

SEQ ID NO: 16 - DNA, 21 bp, Artificial
  source (1)..(21): Description of artificial sequence DNA target sequence
aattctccgaacgtgtcacgt

SEQ ID NO: 17 - DNA, 21 bp, Artificial
  source (1)..(21): Description of artificial sequence dsRNA
uucuccgaacgugucacgutt

SEQ ID NO: 18 - DNA, 21 bp, Artificial
  source (1)..(21): Description of artificial sequence dsRNA
acgugacacguucggagaatt